(19) United States
(12) Patent Application Publication
(10) Pub. No.: US 2008/0318845 A1
(43) Pub. Date: Dec. 25, 2008

(54) Selective Vpac2 Receptor Peptide Agonists

(75) Inventors: Bengt Krister Bokvist, Hamburg (DE); Lianshan Zhang, Carmel, IN (US); Jorge Alsina-Fernandez, Indianapolis, IN (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, Indianapolis, IN (US)

(21) Appl. No.: 11/573,903

(22) PCT Filed: Aug. 11, 2005
(86) PCT No.: PCT/US05/28532

§ 371 Date: Feb. 19, 2007

Related U.S. Application Data
(60) Provisional application No. 60/602,350, filed on Aug. 18, 2004.
(60) Provisional application No. 60/602,461, filed on Aug. 18, 2004.

(51) Int. Cl. A61K 38/17; C07K 14/47; A61P 3/10

(52) U.S. Cl. 514/11; 530/317

(57) ABSTRACT

The present invention relates to peptides that selectively activate the VPAC2 receptor and are useful in the treatment of diabetes.

The present invention relates to selective VPAC2 receptor peptide agonists.

More particularly, this invention is directed to selective VPAC2 receptor peptide agonists which are cyclic.

Type 2 diabetes, or non-insulin dependent diabetes mellitus (NIDDM), is the most common form of diabetes, affecting 90% of people with diabetes. With NIDDM, patients have impaired β-cell function resulting in insufficient insulin production and/or decreased insulin sensitivity. If NIDDM is not controlled, excess glucose accumulates in the blood, resulting in hyperglycemia. Over time, more serious complications may arise including renal dysfunction, cardiovascular problems, visual loss, lower limb ulceration, neuropathy, and ischemia. Treatments for NIDDM include improving diet, exercise, and weight control as well as using a variety of oral medications. Individuals with NIDDM can initially control their blood glucose levels by taking such oral medications. These medications do not, however, slow the progressive loss of β-cell function that occurs in type 2 diabetes patients and, thus, are not sufficient to control blood glucose levels in the later stages of the disease. Also, treatment with currently available medications exposes NIDDM patients to potential side effects such as hypoglycemia, gastrointestinal problems, fluid retention, oedema, and/or weight gain.

Compounds, such as peptides that are selective for a particular G-protein coupled receptor known as the VPAC2 receptor, were initially identified by modifying vasoactive intestinal peptide (VIP) and/or pituitary adenylate cyclase-activating polypeptide (PACAP). (See, for example, Xia et al., J Pharmacol Exp Ther., 281:629-633 (1997); Tsutsumi et al., Diabetes, 51:1453-1460 (2002), WO 01/23420, WO 2004/006839).

PACAP belongs to the secretin/glucagon/vasoactive intestinal peptide (VIP) family of peptides and works through three G-protein-coupled receptors that exert their action through the cAMP-mediated and other Ca²⁺-mediated signal transduction pathways. These receptors are known as the PACAP-preferring type 1 (PAC1) receptor (Isobe, et al., Regul. Pept., 110:213-217 (2003); Ogi, et al., Biochem. Biophys. Res. Commun., 196:1511-1521 (1993)) and the two VIP-shared type 2 receptors (VPAC1 and VPAC2) (Sherwood et al., Endocr. Rev., 21:619-670 (2000); Hammar et al., Pharmacol Rev, 50:265-270 (1998); Couvineau, et al., J. Biol. Chem., 278:24759-24766 (2003); Sreedharan, et al., Biochem. Biophys. Res. Commun., 193:546-553 (1993); Lutz, et al., FEBS Lett., 458: 197-203 (1999); Adamou, et al., Biochem. Biophys. Res. Commun., 209: 385-392 (1995)).

PACAP has comparable activities towards all three receptors, whilst VIP selectively activates the two VPAC receptors (Tsutsumi et al., Diabetes, 51:1453-1460 (2002)). Both VIP (Eriksson et al., Peptides, 10: 481-484 (1989)) and PACAP (Filipsson et al., JCEM, 82:3093-3098 (1997)) have been shown to not only stimulate insulin secretion in man when given intravenously but also increase glucagon secretion and hepatic glucose output. As a consequence, PACAP or VIP stimulation generally does not result in a net improvement of glycemia. Activation of multiple receptors by PACAP or VIP also has broad physiological effects on nervous, endocrine, cardiovascular, reproductive, muscular, and immune systems (Gozes et al., Curr. Med. Chem., 6:1019-1034 (1999)). Furthermore, it appears that VIP-induced watery diarrhoea in rats is mediated by only one of the VPAC receptors, VPAC1 (Ito et al., Peptides, 22:1139-1151 (2001); Tsutsumi et al., Diabetes, 51:1453-1460 (2002)). In addition, the VPAC1 and PAC1 receptors are expressed on α-cells and hepatocytes and, thus, are most likely involved in the effects on hepatic glucose output.

Known natural VIP related peptides include helodermin and helospectin, which are isolated from the salivary excretions of the Gila Monster (Heloderma Suspectum). The main difference between helodermin and helospectin is the presence in helodermin of two consecutive acidic residues in positions 8 and 9. The different behaviour of helodermin and helospectin in rat and human is of particular interest as lizard peptides are long acting VIP analogues.

WO 91/06565 (Diacel Chemical Industries and Meiji Seika Kaisha Ltd) describes three peptides having an activity of relaxing smooth or unstriated muscles. Described are peptides, which include a helodermin derivative comprising a combination of the amino acid sequence of VIP with a part of the amino acid sequence of helodermin, as well as a peptide composed of a combination of a part of the amino acid sequence of VIP with another part of the amino acid sequence of helodermin.

Exendin-4 is also found in the salivary excretions from the Gila Monster, Heloderma Suspectum, (Eng et al., J. Biol. Chem., 267(11):7402-7405 (1992)). It is a 39 amino acid peptide, which has glucose dependent insulin secretagogue activity.

Information obtained from studying the structure and proteolytic cleavage of linear VIP analogues has been used in the synthesis and development of cyclic VIP analogues (Bolin et al., Biopolymers (Peptide Science), 37:57-66 (1995) and Bolin et al., Drug Design and Discovery, 13:107-114 (1996)). U.S. Pat. No. 5,677,419 and EP 0 536 741 (Hoffmann-La Roche Inc.) disclose a series of cyclised VIP analogues, which are useful for the treatment of asthma. A process for the synthesis of a cyclic VIP analogue from four protected peptides fragments is described in U.S. Pat. No. 6,080,837 (also, U.S. Pat. No. 6,316,593) and WO 97/29126 (Hoffmann-La Roche Inc.). One particular cyclic VIP analogue, identified as RO 15-1392, has been shown to be a selective VPAC2 receptor agonist (Bolin et al., J. Pharmacol. Exp. Ther., 281(2):629-633 (1997)). In addition, a cyclic VIP analogue was used as the starting point for the development of a VPAC2 receptor peptide antagonist (Moreno et al., Peptides, 21:1543-1549 (2000)).

Recent studies have shown that peptides selective for the VPAC2 receptor are able to stimulate insulin secretion from the pancreas without gastrointestinal (GI) side effects and without enhancing glucagon release and hepatic glucose output (Tsutsumi et al., Diabetes, 51:1453-1460 (2002)).

Many of the VPAC2 receptor peptide agonists reported to date, however, have less than desirable potency, selectivity, and stability profiles, which could impede their clinical viability. In addition, many of these peptides are not suitable for commercial candidates as a result of stability issues associated with the polypeptides in formation, as well as issues with the short half-life of these polypeptides in vivo. There is, therefore, a need for new therapies, which overcome the problems associated with current medications for NIDDM.

The present invention seeks to provide improved compounds that are selective for the VPAC2 receptor and which induce insulin secretion from the pancreas only in the presence of high blood glucose levels. The compounds of the present invention are peptides, which are believed to also improve beta cell function. These peptides can have the physiological effect of inducing insulin secretion without GI side effects or a corresponding increase in hepatic glucose output and also generally have enhanced selectivity, potency, and/or in vivo stability of the peptide compared to known VPAC2 receptor peptide agonists.

The present invention particularly seeks to provide cyclic VPAC2 receptor peptide agonists having increased selectivity, potency and/or stability compared to linear VPAC2 receptor peptide agonists.

According to a first aspect of the present invention, there is provided a cyclic VPAC2 receptor peptide agonist comprising a sequence of the formula:

Formula 1

(SEQ ID NO: 1)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂-Xaa₃₃-Xaa₃₄-
Xaa₃₅-Xaa₃₆-Xaa₃₇-Xaa₃₈-Xaa₃₉-Xaa₄₀

wherein:
Xaa₁ is: His, dH, or is absent;

### Xaa₂ is: dA, Ser, Val, Gly, Thr, Leu, dS, Pro, or Aib;

### Xaa₃ is: Asp or Glu;

### Xaa₄ is: Ala, Ile, Tyr, Phe, Val, Thr, Leu, Trp, Gly, dA, Aib, or NMeA;

### Xaa₅ is: Val, Leu, Phe, Ile, Thr, Trp, Tyr, dV, Aib, or NMeV;

### Xaa₆ is: Phe, Ile, Leu, Thr, Val, Trp, or Tyr;

### Xaa₈ is: Asp, Glu, Ala, Lys, Leu, Arg, Tyr, Orn, or Dab;

### Xaa₉ is: Asn, Gln, Asp, Glu, Ser, Cys, or hC;

### Xaa₁₀ is: Tyr, Trp, or Tyr(OMe);

Xaa₁₂ is: Arg, Lys, Glu, hR, Orn, Lys (isopropyl), Aib, Cit, Ala, Leu, Gln, Phe, Cys, hC, Asp, or Dab;

### Xaa₁₃ is: Leu, Phe, Glu, Ala, Aib, Ser, Cys, hC, Asp, or K(CO(CH₂)₂SH);

Xaa₁₄ is: Arg, Leu, Lys, Ala, hR, Orn, Lys (isopropyl), Phe, Gln, Aib, Cit, or Dab;
Xaa₁₅ is: Lys, Ala, Arg, Glu, Leu, hR, Orn, Lys (isopropyl), Phe, Gln, Aib, K(Ac), Cit, Asp, Dab, or K(W);
Xaa₁₆ is: Gln, Lys, Glu, Ala, hR, Orn, Lys (isopropyl), Cit, Ser, Cys, hC, Asp, Dab, or K(CO(CH₂)₂SH);

### Xaa₁₇ is: Val, Ala, Leu, Ile, Met, Nle, Lys, Aib, Ser, Cys, hC, Orn, Dab, or K(CO(CH₂)₂SH);

### Xaa₁₈ is: Ala, Ser, Cys, or hC;

### Xaa₁₉ is: Val, Ala, Glu, Phe, Gly, His, Ile, Lys, Leu, Met, Asn, Pro, Gln, Arg, Ser, Thr, Trp, Tyr, Cys, Asp, Orn, Dab, hC, or K(CO(CH₂)₂SH);

Xaa₂₀ is: Lys, Gln, hR, Arg, Ser, His, Orn, Lys (isopropyl), Ala, Aib, Trp, Thr, Leu, Ile, Phe, Tyr, Val, K(Ac), Cit, Cys, hC, or Dab;

### Xaa₂₁ is: Lys, His, Arg, Ala, Phe, Aib, Leu, Gln, Orn, hR, K(Ac), Cit, Ser, Cys, hC, Dab, Val, Tyr, Ile, Thr, Trp, Asp, or Glu;

### Xaa₂₂ is: Tyr, Trp, Phe, Thr, Leu, Ile, Val, Tyr(OMe), Ala, Aib, Ser, Cys, or hC;

### Xaa₂₃ is: Leu, Phe, Ile, Ala, Trp, Thr, Val, Aib, Ser, Cys, or hC;

### Xaa₂₄ is: Gln, Glu, Asn, Ser, Cys, hC, Asp, or K(CO(CH₂)₂SH);

### Xaa₂₅ is: Ser, Asp, Phe, Ile, Leu, Thr, Val, Trp, Gln, Asn, Tyr, Aib, Glu, Cys, hC, Lys, Orn, Dab, or K(CO(CH₂)₂SH);

### Xaa₂₆ is: Ile, Leu, Thr, Val, Trp, Tyr, Phe, Aib, Ser, Cys, hC, or K(CO(CH₂)₂SH);

Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, Leu, Orn, dK, hC, or Dab;
Xaa₂₈ is: Asn, Asp, Gln, Lys, Arg, Aib, Orn, hR, Cit, Pro, dK, Glu, Dab, Cys, hC, K(CO(CH₂)₂SH), or is absent;
Xaa₂₉ is: Lys, Ser, Arg, Asn, hR, Ala, Asp, Glu, Phe, Gly, His, Ile, Leu, Met, Pro, Gln, Thr, Val, Trp, Tyr, Cys, Orn, Cit, Aib, Dab, hC, or is absent;
Xaa₃₀ is: Arg, Lys, Ile, Ala, Asp, Glu, Phe, Gly, His, Leu, Met, Asn, Pro, Gln, Ser, Thr, Val, Trp, Tyr, Cys, hR, Cit, Aib, Orn, Dab, hC, or is absent;
Xaa₃₁ is: Tyr, His, Phe, Thr, Cys, Gln, hC, or is absent;
Xaa₃₂ is: Ser, Cys, hC, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₈, Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the peptide agonist sequence,

and a C-terminal extension wherein the N-terminus of the C-terminal extension is linked to the C-terminus of the peptide of Formula 1,

wherein the C-terminal extension comprises an amino acid sequence of the formula:

Formula 2

(SEQ ID NO: 2)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, or absent;
Xaa₂ is: Gly, Arg, Cys, or absent;
Xaa₃ is: Pro, Thr, Ser, Ala, Cys, or absent;
Xaa₄ is: Ser, Pro, His, Cys, or absent;
Xaa₅ is: Ser, Arg, Thr, Trp, Lys, Cys, or absent;
Xaa₆ is: Gly, Ser, Cys, or absent;
Xaa₇ is: Ala, Asp, Arg, Glu, Lys, Gly, Cys, or absent;
Xaa₈ is: Pro, Ser, Ala, Cys, or absent;
Xaa₉ is: Pro, Ser, Ala, Cys, or absent;
Xaa₁₀ is: Pro, Ser, Ala, Arg, Lys, Hiss Cys, or absent;
Xaa₁₁ is: Ser, Cys, His, Pro, Lys, Arg, or absent;
Xaa₁₂ is: His, Ser, Arg, Lys, Cys, or absent; and
Xaa₁₃ is: His, Ser, Arg, Lys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated,

or wherein the C-terminal extension comprises an amino acid sequence of the formula:

Formula 3

(SEQ ID NO: 3)

Xaa₁-Xaa₂-Xaa₃-Xaa₄-Xaa₅-Xaa₆-Xaa₇-Xaa₈-Xaa₉-Xaa₁₀

wherein:
Xaa₁ is: Ser, or absent;
Xaa₂ is: Arg, Ser, hR, Orn, His, or absent;
Xaa₃ is: Thr, or absent;
Xaa₄ is: Ser, or absent;
Xaa₅ is: Pro, Ser, Ala, or absent;
Xaa₆ is: Pro, Ser, Ala, Arg, or absent;
Xaa₇ is: Pro, Ser, Ala, or absent;
Xaa₈ is: Lys, K(W), Pro, or absent;
Xaa₉ is: K(E-C₁₆), Ser, or absent; and
Xaa₁₀ is: Ser, or absent;

provided that at least three of Xaa₁ to Xaa₁₀ of the C-terminal extension are present and provided that if Xaa₁, Xaa₂, Xaa₃, Xaa₄, Xaa₅, Xaa₆, Xaa₇, Xaa₈, or Xaa₉ is absent, the next amino acid present downstream is the next amino acid in the C-terminal extension and wherein the C-terminal amino acid may be amidated.

Preferably, the cyclic VPAC2 receptor peptide agonist comprises a sequence of the formula:

Formula 4

(SEQ ID NO: 4)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂

wherein:
Xaa₁ is: His, dH, or is absent;

### Xaa₂ is: dA, Ser, Val, Gly, Thr, Leu, dS, Pro, or Aib;

### Xaa₃ is: Asp or Glu;

### Xaa₄ is: Ala, Ile, Tyr, Phe, Val, Thr, Leu, Trp, Gly, dA, Aib, or NMeA;

### Xaa₅ is: Val, Leu, Phe, Ile, Thr, Trp, Tyr, dV, Aib, or NMeV;

### Xaa₆ is: Phe, Ile, Leu, Thr, Val, Trp, or Tyr;

### Xaa₈ is: Asp, Glu, Ala, Lys, Leu, Arg, Tyr, Orn, or Dab;

### Xaa₉ is: Asn, Gln, Glu, Ser, Cys, hC, or Asp;

### Xaa₁₀ is: Tyr, Trp, or Tyr(OMe);

### Xaa₁₂ is: Arg, Lys, hR, Orn, Aib, Cit, Ala, Leu, Gln, Phe, Cys, hC, or Dab;

### Xaa₁₃ is: Leu, Phe, Glu, Ala, Aib, Ser, Cys, hC, Asp, or K(CO(CH₂)₂SH);

### Xaa₁₄ is: Arg, Leu, Lys, Ala, hR, Orn, Phe, Gln, Aib, Cit, or Dab;

### Xaa₁₅ is: Lys, Ala, Arg, Glu, Leu, hR, Orn, Phe, Gln, Aib, K(Ac), Cit, Asp, Dab, or K(W);

### Xaa₁₆ is: Gln, Lys, Ala, hR, Orn, Cit, Ser, Cys, hC, Dab, or K(CO(CH₂)₂SH);

### Xaa₁₇ is: Val, Ala, Leu, Ile, Met, Nle, Lys, Aib, Ser, Cys, hC, Orn, Dab, or K(CO(CH₂)₂SH);

### Xaa₁₈ is: Ala, Ser, Cys, or hC;

### Xaa₁₉ is: Ala, Gly, Leu, Ser, Cys, hC, or K(CO(CH₂)₂SH);

### Xaa₂₀ is: Lys, Gln, hR, Arg, Ser, Orn, Ala, Aib, Trp, Thr, Leu, Ile, Phe, Tyr, Val, K(Ac), Cit, Cys, hC, or Dab;

### Xaa₂₁ is: Lys, Arg, Ala, Phe, Aib, Leu, Gln, Orn, hR, K(Ac), Cit, Ser, Cys, hC, Dab, Asp, or Glu;

### Xaa₂₂ is: Tyr, Trp, Phe, Thr, Leu, Ile, Val, Tyr(OMe), Ala, Aib, Ser, Cys, or hC;

### Xaa₂₃ is: Leu, Phe, Ile, Ala, Trp, Thr, Val, Aib, Ser, Cys, or hC;

### Xaa₂₄ is: Gln, Asn, Ser, Cys, hC, or K(CO(CH₂)₂SH);

### Xaa₂₅ is: Ser, Asp, Phe, Ile, Leu, Thr, Val, Trp, Gln, Asn, Tyr, Aib, Glu, Cys, hC, Lys, Orn, Dab, or K(CO(CH₂)₂SH);

### Xaa₂₆ is: Ile, Leu, Thr, Val, Trp, Tyr, Phe, Aib, Ser, Cys, hC, or K(CO(CH₂)₂SH);

### Xaa₂₇ is: Lys, hR, Arg, Gln, Orn, dK, or Dab;

Xaa₂₈ is: Asn, Gln, Lys, Arg, Aib, Orn, hR, Cit, Pro, dK, Dab, Cys, hC, K(CO(CH₂)₂SH), or is absent;
Xaa₂₉ is: Lys, Ser, Arg, Asn, hR, Orn, Cit, Aib, Dab, or is absent;
Xaa₃₀ is: Arg, Lys, Ile, hR, Cit, Aib, Orn, Dab, or is absent;
Xaa₃₁ is: Tyr, His, Phe, Gln, or is absent; and
Xaa₃₂ is: Cys, hC, or is absent;

provided that if Xaa₂₈, Xaa₂₉, Xaa₃₀, or Xaa₃₁ is absent, the next amino acid present downstream is the next amino acid in the peptide agonist sequence,

and a C-terminal extension wherein the N-terminus of the C-terminal extension is linked to the C-terminus of the peptide of Formula 4,

wherein the C-terminal extension comprises an amino acid sequence of the formula:

Formula 2

(SEQ ID NO: 2)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, or absent;
Xaa₂ is: Gly, Arg, Cys, or absent;
Xaa₃ is: Pro, Thr, Ser, Ala, Cys, or absent;
Xaa₄ is: Ser, Pro, His, Cys, or absent;
Xaa₅ is: Ser, Arg, Thr, Trp, Lys, Cys, or absent;
Xaa₆ is: Gly, Ser, Cys, or absent;
Xaa₇ is: Ala, Asp, Arg, Glu, Lys, Gly, Cys, or absent;
Xaa₈ is: Pro, Ser, Ala, Cys, or absent;
Xaa₉ is: Pro, Ser, Ala, Cys, or absent;
Xaa₁₀ is: Pro, Ser, Ala, Arg, Lys, His, Cys, or absent;
Xaa₁₁ is: Ser, Cys, His, Pro, Lys, Arg, or absent;
Xaa₁₂ is: His, Ser, Arg, Lys, Cys, or absent; and
Xaa₁₃ is: His, Ser, Arg, Lys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated,

or wherein the C-terminal extension comprises an amino acid sequence the formula:

Formula 3

(SEQ ID NO: 3)

Xaa₁-Xaa₂-Xaa₃-Xaa₄-Xaa₅-Xaa₆-Xaa₇-Xaa₈-Xaa₉-Xaa₁₀

wherein:
Xaa₁ is: Ser, or absent;
Xaa₂ is: Arg, Ser, hR, Orn, His, or absent;
Xaa₃ is: Thr, or absent;
Xaa₄ is: Ser, or absent;
Xaa₅ is: Pro, Ser, Ala, or absent;
Xaa₆ is: Pro, Ser, Ala, Arg, or absent;
Xaa₇ is: Pro, Ser, Ala, or absent;
Xaa₈ is: Lys, K(W), Pro, or absent;
Xaa₉ is: K(E-C₁₆), Ser, or absent; and
Xaa₁₀ is: Ser, or absent;

provided that at least three of Xaa₁ to Xaa₁₀ of the C-terminal extension are present and provided that if Xaa₁, Xaa₂, Xaa₃, Xaa₄, Xaa₅, Xaa₆, Xaa₇, Xaa₈, or Xaa₉ is absent, the next amino acid present downstream is the next amino acid in the C-terminal extension and wherein the C-terminal amino acid may be amidated.

The cyclic VPAC2 receptor peptide agonist more preferably comprises a sequence of the formula:

Formula 5

(SEQ ID NO: 5)

His-Ser-Xaa₃-Ala-Val-Phe-Thr-Xaa₈-Asn-Tyr(OMe)-
Thr-Xaa₁₂-Xaa₁₃-Xaa₁₄-Xaa₁₅-Xaa₁₆-Nle-Ala-Ala-
Xaa₂₀-Xaa₂₁-Tyr-Leu-Asn-Xaa₂₅-Xaa₂₆-Xaa₂₇-Xaa₂₈-
Xaa₂₉

wherein:

### Xaa₃ is: Asp, or Glu;

### Xaa₈ is: Asp, or Glu;

### Xaa₁₂ is: Lys, Cys, hC, hR, Orn, or Dab;

### Xaa₁₃ is: Leu, or Aib;

### Xaa₁₄ is: Arg, or Aib;

### Xaa₁₅ is: Lys, Orn, Dab, or Aib;

### Xaa₁₆ is: Glu, Cys, or hC;

### Xaa₂₀ is: Lys, hR, Orn, or Dab;

### Xaa₂₁ is: Lys, Cys, hR, hC, Orn, or Dab;

### Xaa₂₅ is: Ser, Cys, Asp, hC, or Glu;

### Xaa₂₆ is: Leu, or Ile;

### Xaa₂₇ is: Lys, hR, Orn, or Dab;

### Xaa₂₈ is: Lys, Asn, hR, Gln, Aib, Orn, Dab, or Pro; and

Xaa₂₉ is: Lys, Orn, Dab, hR, or is absent;

and a C-terminal extension wherein the N-terminus of the C-terminal extension is linked to the C-terminus of the peptide of Formula 5,

wherein the C-terminal extension comprises an amino acid sequence of the formula:

Formula 2

(SEQ ID NO: 2)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, or absent;
Xaa₂ is: Gly, Arg, Cys, or absent;
Xaa₃ is: Pro, Thr, Ser, Ala, Cys, or absent;
Xaa₄ is: Ser, Pro, His, Cys, or absent;
Xaa₅ is: Ser, Arg, Thr, Trp, Lys, Cys, or absent;
Xaa₆ is: Gly, Ser, Cys, or absent;
Xaa₇ is: Ala, Asp, Arg, Glu, Lys, Gly, Cys, or absent;
Xaa₈ is: Pro, Ser, Ala, Cys, or absent;
Xaa₉ is: Pro, Ser, Ala, Cys, or absent;
Xaa₁₀ is: Pro, Ser, Ala, Arg, Lys, His, Cys, or absent;
Xaa₁₁ is: Ser, Cys, His, Pro, Lys, Arg, or absent;
Xaa₁₂ is: His, Ser, Arg, Lys, Cys, or absent; and
Xaa₁₃ is: His, Ser, Arg, Lys, or absent;

provided that at least five of Xaa₁ to Xaa₁₃ of the C-terminal extension are present and provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated,

or wherein the C-terminal extension comprises an amino acid sequence of the formula:

Formula 3

(SEQ ID NO: 3)

Xaa₁-Xaa₂-Xaa₃-Xaa₄-Xaa₅-Xaa₆-Xaa₇-Xaa₈-Xaa₉-Xaa₁₀

wherein:
Xaa₁ is: Ser, or absent;
Xaa₂ is: Arg, Ser, hR, Orn, His, or absent;
Xaa₃ is: Thr, or absent;
Xaa₄ is: Ser, or absent;
Xaa₅ is: Pro, Ser, Ala, or absent;
Xaa₆ is: Pro, Ser, Ala, Arg, or absent;
Xaa₇ is: Pro, Ser, Ala, or absent;
Xaa₈ is: Lys, K(W), Pro, or absent;
Xaa₉ is: K(E-C₁₆), Ser, or absent; and
Xaa₁₀ is: Ser, or absent;

provided that at least three of Xaa₁ to Xaa₁₀ of the C-terminal extension are present and provided that if Xaa₁, Xaa₂, Xaa₃, Xaa₄, Xaa₅, Xaa₆, Xaa₇, Xaa₈, or Xaa₉ is absent, the next amino acid present downstream is the next amino acid in the C-terminal extension and wherein the C-terminal amino acid may be amidated.

Preferably, at least six of Xaa₁ to Xaa₁₃ of the C-terminal extension in Formula 2 are present. More preferably, at least seven, eight, nine, ten, eleven, twelve or all of Xaa₁ to Xaa₁₃ of the C-terminal extension are present

Preferably, at least four of Xaa₁ to Xaa₁₀ of the C-terminal extension in Formula 3 are present. More preferably, at least five, six, seven, eight, nine or all of Xaa₁ to Xaa₁₀ of the C-terminal extension are present

Preferably, the cyclic VPAC2 receptor peptide agonist is cyclised by means of a lactam bridge. It is preferred that the lactam bridge is formed by the covalent attachment of the side chain of the residue at Xaaₙ to the side chain of the residue at Xaaₙ₊₄, wherein n is 1 to 28. Preferably, n is 12, 20, or 21. More preferably, n is 21. It is also preferred that the lactam bridge is formed by the covalent attachment of the side chain of a Lys, Orn or Dab residue to the side chain of an Asp or Glu residue.

The cyclic VPAC2 receptor peptide agonist may alternatively be cyclised by means of a disulfide bride. It is preferred that the disulfide bridge is formed by the covalent attachment of the side chain of the residue at Xaaₙ to the side chain of the residue at Xaaₙ₊₄, wherein n is 1 to 30 and is preferably 1 to 28. Even more preferably, n is 12, 20, or 21. It is also preferred that the disulfide bridge is formed by the covalent attachment of the side chain of a Cys or hC residue to the side chain of another Cys or hC residue.

Alternatively, the lactam bridge or the disulfide bridge may be formed by the covalent attachment of the side chain of the residue at Xaaₙ to the side chain of the residue at Xaaₙ₊₃, wherein n is 1 to 28. The lactam bridge or the disulfide bridge may also be formed by the covalent attachment of the side chain of the residue at Xaaᵢ to the side chain of the residue at Xaaᵢ₊₇ or Xaaᵢ₊₈, wherein i is 1 to 24.

Preferably, the C-terminal extension of the cyclic VPAC2 receptor peptide agonist comprises an amino acid sequence of the formula:

Formula 6

(SEQ ID NO: 6)

Xaa₁-Xaa₂-Xaa₃-Xaa₄-Xaa₅-Xaa₆-Xaa₇-Xaa₈-Xaa₉-
Xaa₁₀-Xaa₁₁

wherein:
Xaa₁ is: Gly, Cys, or absent;
Xaa₂ is: Gly, Arg, or absent;
Xaa₃ is: Pro, Thr, or absent;
Xaa₄ is: Ser, or absent;
Xaa₅ is: Ser, or absent;
Xaa₆ is: Gly, or absent;
Xaa₇ is: Ala, or absent;
Xaa₈ is: Pro, or absent;
Xaa₉ is: Pro, or absent;
Xaa₁₀ is: Pro, or absent; and
Xaa₁₁ is: Ser, Cys, or absent;

provided that at least five of Xaa₁ to Xaa₁₁ of the C-terminal extension are present and provided that if Xaa₁, Xaa₂, Xaa₃, Xaa₄, Xaa₅, Xaa₆, Xaa₇, Xaa₈, Xaa₉, or Xaa₁₀ is absent, the next amino acid present downstream is the next amino acid in the C-terminal extension and wherein the C-terminal amino acid may be amidated.

Preferably, at least six of Xaa₁ to Xaa₁₁ of the C-terminal extension in Formula 6 are present. More preferably at least seven, eight, nine, ten or all of Xaa₁ to Xaa₁₁ of the C-terminal extension are present

More preferably, the C-terminal extension of the cyclic VPAC2 receptor peptide agonist is selected from:

| | |
|---|---|
| SEQ ID NO: 10 | GGPSSGAPPPS |
| SEQ ID NO: 11 | GGPSSGAPPPS-NH₂ |
| SEQ ID NO: 12 | GGPSSGAPPPC |
| SEQ ID NO: 13 | GGPSSGAPPPC-NH₂ |
| SEQ ID NO: 14 | GRPSSGAPPPS |
| SEQ ID NO: 15 | GRPSSGAPPPS-NH₂ |

Alternatively, the C-terminal extension of the cyclic VPAC2 receptor peptide agonist may comprise an amino acid sequence of the formula:

Formula 7

(SEQ ID NO: 7)

Xaa₁-Xaa₂-Xaa₃-Xaa₄-Xaa₅-Xaa₆-Xaa₇-Xaa₈-Xaa₉

wherein:
Xaa₁ is: Ser or absent;
Xaa₂ is: Arg, or absent;
Xaa₃ is: Thr or absent;
Xaa₄ is: Ser or absent;
Xaa₅ is: Pro or absent;
Xaa₆ is: Pro or absent;
Xaa₇ is: Pro or absent;
Xaa₈ is: Lys, K(W), or absent; and
Xaa₉ is: K(E-Cl₆) or absent;

provided that at least three of Xaa₁ to Xaa₉ of the C-terminal extension are present and provided that if Xaa₁, Xaa₂, Xaa₃, Xaa₄, Xaa₅, Xaa₆, Xaa₇, or Xaa₈ is absent, the next amino acid present downstream is the next amino acid in the C-terminal extension and wherein the C-terminal amino acid may be amidated.

Preferably, at least four of Xaa₁ to Xaa₉ of the C-terminal extension in Formula 7 are present. More preferably, at least five, six, seven, eight, or all of Xaa₁ to Xaa₉ of the C-terminal extension are present

More preferably, C-terminal extension of the cyclic VPAC2 receptor peptide agonist is selected from:

| | |
|---|---|
| SEQ ID NO: 16 | SRTSPPP |
| SEQ ID NO: 17 | SRTSPPP-NH₂ |
| SEQ ID NO: 18 | SSTSPRPPSS |
| SEQ ID NO: 19 | SSTSPRPPSS-NH₂ |

Preferably, the cyclic VPAC2 receptor peptide agonist comprises a sequence of the Formula 1 (SEQ ID NO: 1), Formula 4 (SEQ ID NO: 4) or Formula 5 (SEQ ID NO: 5) wherein Xaa₁₂ is Lys, Orn, or hR, Xaa₁₃ is Leu, or Aib, Xaa₁₅ is Lys, Aib, or Orn, Xaa₂₀ is Lys, or Orn, Xaa₂₇ is Lys, Orn, or hR, Xaa₂₈ is Lys, Orn, Aib, Gln, hR, or Pro, and Xaa₂₉ is Orn, Lys, hR, or absent. Preferably, Xaa₃₀ and all subsequent residues in Formula 1 (SEQ ID NO: 1), Formula 4 (SEQ ID NO: 4) or Formula 5 (SEQ ID NO: 5) are absent.

Preferably, the cyclic VPAC2 receptor peptide agonist sequence further comprises a histidine residue at the N-terminal extension region of the peptide sequence before Xaa₁

Preferably, the cyclic VPAC2 receptor peptide agonist further comprises a N-terminal modification at the N-terminus of the peptide agonist wherein the N-terminal modification is selected from:
(a) addition of D-histidine, isoleucine, methionine, or norleucine;
(b) addition of a peptide comprising the sequence Ser-Trp-Cys-Glu-Pro-Gly-Trp-Cys-Arg (SEQ ID NO: 20) wherein the Arg is linked to the N-terminus of the peptide agonist;
(c) addition of C₁-C₁₆ alkyl optionally substituted with one or more substituents independently selected from aryl, C₁-C₆ alkoxy, —NH₂, —OH, halogen and —CF₃;
(d) addition of —C(O)R¹ wherein R¹ is a C₁-C₁₆ alkyl optionally substituted with one or more substituents independently selected from aryl, C₁-C₆ alkoxy, —NH₂, —OH, halogen, —SH and —CF₃; a aryl or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; —NR²R³ wherein R² and R³ are independently hydrogen, C₁-C₆ alkyl, aryl or aryl C₁-C₄ alkyl; —OR⁴ wherein R⁴ is C₁-C₁₆ alkyl optionally substituted with one or more substituents independently selected from aryl, C₁-C₆ alkoxy, —NH₂, —OH, halogen and —CF₃, aryl or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; or 5-pyrrolidin-2-one;
(e) addition of —SO₂R⁵ wherein R⁵ is aryl, aryl C₁-C₄ alkyl or C₁-C₁₆ alkyl;
(f) formation of a succinimide group optionally substituted with C₁-C₆ alkyl or —SR⁶, wherein R⁶ is hydrogen or C₁-C₆ alkyl;
(g) addition of methionine sulfoxide;
(h) addition of biotinyl-6-aminohexanoic acid (6-aminocaproic acid); and
(i) addition of —C(═NH)—NH₂.

More preferably, the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂. Even more preferably, the N-terminal modification is the addition of acetyl, hexanoyl, cyclohexanoyl, or propionyl.

It will be appreciated by the person skilled in the art that cyclic VPAC2 receptor peptide agonists comprising various combinations of peptide sequence according to Formula 1, 4, or 5, C-terminal extensions and N-terminal modifications as described herein, may be made based on the above disclosure.

According to a second aspect of the invention the preferred cyclic VPAC2 receptor peptide agonists comprise an amino acid sequence selected from:

| Agonist # | Sequence |
|---|---|
| P10-SEQ IDNO: 24 | 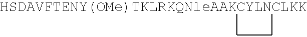 |
| P11-SEQ IDNO: 25 | 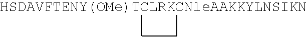 |
| P15-SEQ IDNO: 26 | 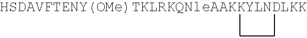 |
| P16-SEQ IDNO: 27 | 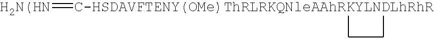 |
| P17-SEQ IDNO: 28 | 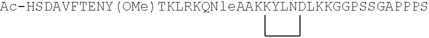 |
| P57-SEQ IDNO: 29 | 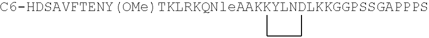 |
| P77-SEQ IDNO: 30 | 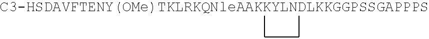 |
| P78-SEQ IDNO: 31 | 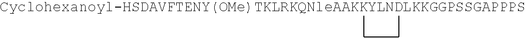 |
| P86-SEQ IDNO: 32 | 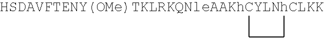 |
| P200-SEQ IDNO: 33 | 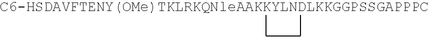 |
| P225-SEQ IDNO: 34 | 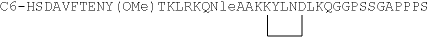 |
| P237-SEQ IDNO: 35 | 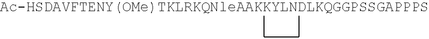 |
| P238-SEQ IDNO: 36 | 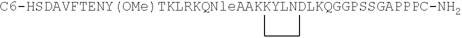 |
| P248-SEQ IDNO: 37 | 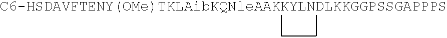 |
| P254-SEQ IDNO: 38 | 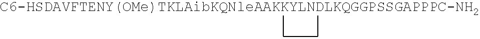 |
| P256-SEQ IDNO: 39 | 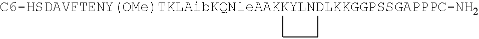 |
| P266-SEQ IDNO: 40 | 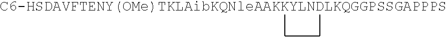 |
| P267-SEQ IDNO: 41 | 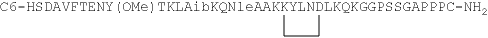 |
| P273-SEQ IDNO: 42 | 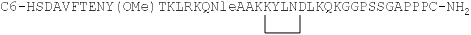 |
| P276-SEQ IDNO: 43 | 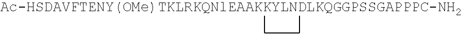 |
| P278-SEQ IDNO: 44 | 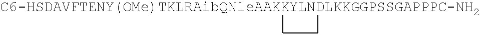 |
| P280-SEQ IDNO: 45 | 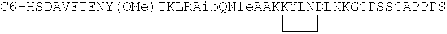 |
| P281-SEQ IDNO: 46 | 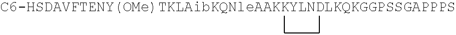 |
| P287-SEQ IDNO: 47 | 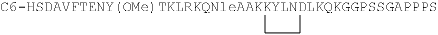 |
| P288-SEQ IDNO: 48 | 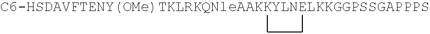 |
| P303-SEQ IDNO: 49 | 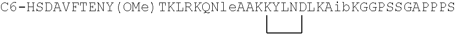 |
| P304-SEQ IDNO: 50 | 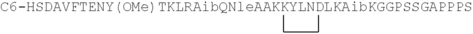 |
| P310-SEQ IDNO: 51 | 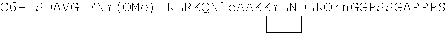 |
| P311-SEQ IDNO: 52 | 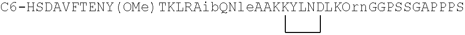 |
| P312-SEQ IDNO: 53 | 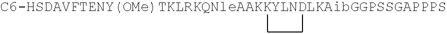 |
| P313-SEQ IDNO: 54 | 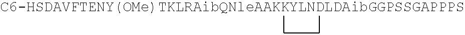 |
| P347-SEQ IDNO: 55 | 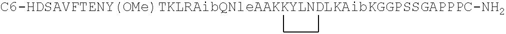 |
| P359-SEQ IDNO: 56 | 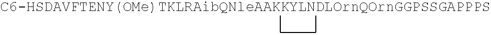 |
| P360-SEQ IDNO: 57 | 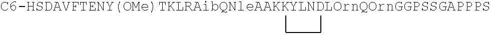 |
| P361-SEQ IDNO: 58 | 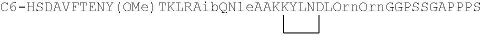 |
| P374-SEQ IDNO: 59 | 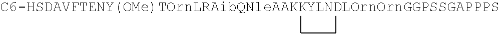 |
| P375-SEQ IDNO: 60 | 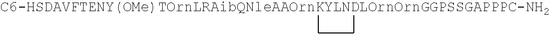 |
| P381-SEQ IDNO: 61 | 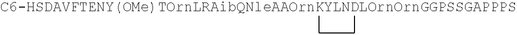 |
| P441-SEQ IDNO: 62 | 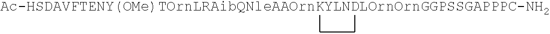 |

More preferred cyclic VPAC2 receptor peptide agonists according to the second aspect of the present invention comprise an amino acid sequence selected from:

| Agonist # | Sequence |
|---|---|
| P17-SEQ IDNO: 28 | 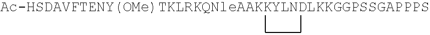 |
| P57-SEQ IDNO: 29 | 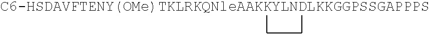 |
| P77-SEQ IDNO: 30 | 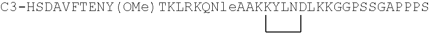 |
| P78-SEQ IDNO: 31 | 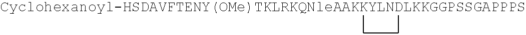 |
| P200-SEQ IDNO: 33 | 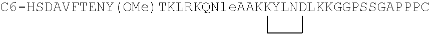 |
| P225-SEQ IDNO: 34 | 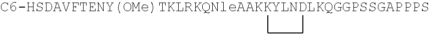 |
| P237-SEQ IDNO: 35 | 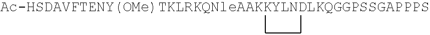 |
| P248-SEQ IDNO: 37 | 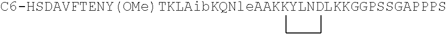 |
| P254-SEQ IDNO: 38 | 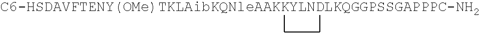 |
| P256-SEQ IDNO: 39 | 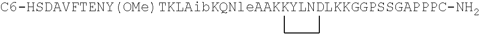 |
| P266-SEQ IDNO: 40 | 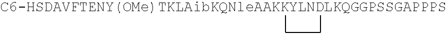 |
| P267-SEQ IDNO: 41 | 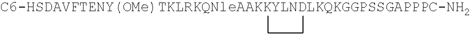 |
| P276-SEQ IDNO: 43 | 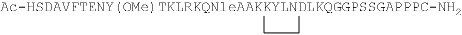 |
| P280-SEQ IDNO: 45 | 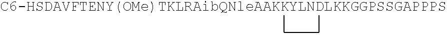 |
| P281-SEQ IDNO: 46 | 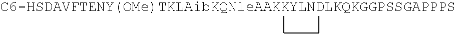 |
| P287-SEQ IDNO: 47 | 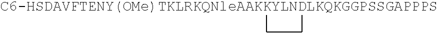 |
| P288-SEQ IDNO: 48 | 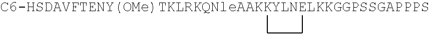 |
| P303-SEQ IDNO: 49 | 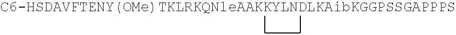 |
| P304-SEQ IDNO: 50 | 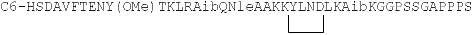 |
| P310-SEQ IDNO: 51 | 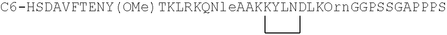 |
| P311-SEQ IDNO: 52 | 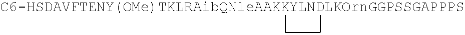 |
| P312-SEQ IDNO: 53 | 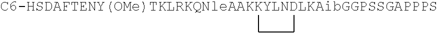 |
| P313-SEQ IDNO: 54 | 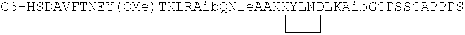 |
| P359-SEQ IDNO: 56 | 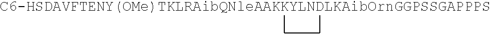 |
| P360-SEQ IDNO: 57 | 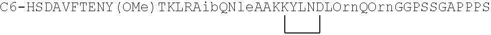 |
| P361-SEQ IDNO: 58 | 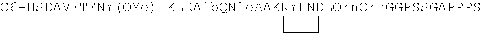 |
| P374-SEQ IDNO: 59 | 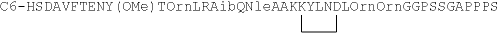 |

According to a third aspect of the invention, there is provided a cyclic VPAC2 receptor peptide agonist comprising a sequence of the formula:

Formula 8

(SEQ ID NO: 8)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂-Xaa₃₃-Xaa₃₄-
Xaa₃₅-Xaa₃₆-Xaa₃₇-Xaa₃₈-Xaa₃₉-Xaa₄₀

wherein:
Xaa₁ is: any naturally occurring amino acid, dH, or is absent;
Xaa₂ is: any naturally occurring amino acid, dA, dS, or Aib;

### Xaa₃ is: Asp or Glu;

Xaa₄ is: any naturally occurring amino acid, dA, Aib, or NMeA;
Xaa₅ is: any naturally occurring amino acid, dV, or Aib;
Xaa₆ is: any naturally occurring amino acid;

### Xaa₈ is: Asp, Glu, Ala, Lys, Leu, Arg, or Tyr;

### Xaa₉ is: Asn, Gln, Asp, Glu, Ser, or Cys;

Xaa₁₀ is: any naturally occurring aromatic amino acid, or Tyr (OMe);
Xaa₁₂ is: hR, Orn, Lys (isopropyl), Aib, Cit, or any naturally occurring amino acid except Pro;
Xaa₁₃ is: Aib, K(CO(CH₂)₂SH), or any naturally occurring amino acid except Pro;
Xaa₁₄ is: hR, Orn, Lys (isopropyl), Aib, Cit, or any naturally occurring amino acid except Pro;
Xaa₁₅ is: hR, Orn, Lys (isopropyl), Aib, K(Ac), Cit, K(W), or any naturally occurring amino acid except Pro;
Xaa₁₆ is: hR, Orn, Lys (isopropyl), Cit, K(CO(CH₂)₂SH), or any naturally occurring amino acid except Pro;
Xaa₁₇ is: Nle, Aib, K(CO(CH₂)₂SH), or any naturally occurring amino acid except Pro;
Xaa₁₈ is: any naturally occurring amino acid;
Xaa₁₉ is: K(CO(CH₂)₂SH), or any naturally occurring amino acid except Pro;
Xaa₂₀ is: hR, Orn, Lys (isopropyl), Aib, K(Ac), Cit, or any naturally occurring amino acid except Pro;
Xaa₂₁ is: hR, Orn, Aib, K(Ac), Cit, or any naturally occurring amino acid except Pro;
Xaa₂₂ is: Aib, Tyr (OMe), or any naturally occurring amino acid except Pro;
Xaa₂₃ is: Aib or any naturally occurring amino acid except Pro;
Xaa₂₄ is: K(CO(CH₂)₂SH), or any naturally occurring amino acid except Pro;
Xaa₂₅ is: Aib, K(CO(CH₂)₂SH), or any naturally occurring amino acid except Pro;
Xaa₂₆ is: K(CO(CH₂)₂SH), or any naturally occurring amino acid except Pro;
Xaa₂₇ is: hR, Lys (isopropyl), Orn, dK, or any naturally occurring amino acid except Pro;
Xaa₂₈ is: any naturally occurring amino acid, Aib, hR, Cit, Orn, dK, or K(CO(CH₂)₂SH);
Xaa₂₉ is: any naturally occurring amino acid, hR, Orn, Cit, Aib, or is absent;
Xaa₃₀ is: any naturally occurring amino acid, hR, Orn, Cit, Aib, or is absent; and
Xaa₃₁ to Xaa₄₀ are any naturally occurring amino acid or are absent;

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈ or Xaa₃₉ is absent, the next amino acid present downstream is the next amino acid in the peptide agonist sequence and that the peptide agonist comprises at least one amino acid substitution selected from:

### Xaa₂ is: dA, Val, Gly, Leu, dS, or Aib;

### Xaa₄ is: Ile, Tyr, Phe, Val, Thr, Leu, Trp, dA, Aib, or NMeA;

### Xaa₅ is: Leu, Phe, Thr, Trp, Tyr, dV, or Aib;

### Xaa₈ is: Leu, Arg, or Tyr;

### Xaa₉ is: Glu, Ser, or Cys;

### Xaa₁₀ is: Trp;

Xaa₁₂ is: Ala, hR, Aib, Lys (isopropyl), Cit, Gln, or Phe;

### Xaa₁₃ is: Phe, Glu, Ala, Aib, Ser, Cys, or K(CO(CH₂)₂SH);

Xaa₁₄ is: Leu, Lys, Ala, hR, Orn, Lys (isopropyl), Phe, Gln, Aib, or Cit;
Xaa₁₅ is: Ala, Arg, Leu, hR, Orn, Lys (isopropyl), Phe, Gln, Aib, K(Ac), Cit, or K(W);
Xaa₁₆ is: Lys, Lys (isopropyl), hR, Orn, Cit, Ser, Cys, or K(CO(CH₂)₂SH);

### Xaa₁₇ is: Lys, Aib, Ser, Cys, or K(CO(CH₂)₂SH);

### Xaa₁₈ is: Ser, or Cys;

### Xaa₁₉ is: K(CO(CH₂)₂SH);

### Xaa₂₀ is: Gln, hR, Arg, Ser, Orn, Lys(isopropyl), Ala, Aib, Trp, Thr, Leu, Ile, Phe, Tyr, Val, K(Ac), Cit, or Cys;

### Xaa₂₁ is: Arg, Ala, Phe, Aib, Leu, Gln, Orn, hR, K(Ac), Cit, Ser, or Cys;

### Xaa₂₂ is: Trp, Thr, Leu, Ile, Val, Tyr(OMe), Ala, Aib, Ser, or Cys;

### Xaa₂₃ is: Phe, Ile, Ala, Trp, Thr, Val, Aib, Ser, or Cys;

### Xaa₂₄ is: Ser, Cys, or K(CO(CH₂)₂SH);

### Xaa₂₅ is: Phe, Ile, Leu, Val, Trp, Gln, Asn, Tyr, Aib, Glu, Cys, or K(CO(CH₂)₂SH);

### Xaa₂₆ is: Thr, Trp, Tyr, Phe, Ser, Cys, or K(CO(CH₂)₂SH);

### Xaa₂₇ is: hR, Orn, or dK;

### Xaa₂₈ is: Pro, Arg, Aib, Orn, hR, Cit, dK, Cys, or K(CO(CH₂)₂SH);

### Xaa₂₉ is: hR, Cys, Orn, Cit, or Aib;

### Xaa₃₀ is: hR, Cit, Aib, or Orn; and

### Xaa₃₁ is: His, or Phe.

Preferably, the VPAC2 receptor peptide agonist according to the third aspect of the present invention comprises a sequence of the formula:

Formula 9

(SEQ ID NO: 9)

His-Xaa₂-Xaa₃-Xaa₄-Xaa₅-Phe-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂-Xaa₃₃-Xaa₃₄-
Xaa₃₅-Xaa₃₆-Xaa₃₇-Xaa₃₈-Xaa₃₉-Xaa₄₀

wherein:

### Xaa₂ is: dA, Ser, Val, Gly, Thr, Leu, dS, Pro, or Aib;

### Xaa₃ is: Asp or Glu;

### Xaa₄ is: Ala, Ile, Tyr, Phe, Val, Thr, Leu, Trp, Gly, dA, Aib, or NMeA;

### Xaa₅ is: Val, Leu, Phe, Ile, Thr, Trp, Tyr, dV, or Aib;

### Xaa₈ is: Asp, Glu, Ala, Lys, Leu, Arg, or Tyr;

### Xaa₉ is: Asn, Gln, Asp, Glu, Ser, or Cys;

### Xaa₁₀ is: Tyr, Trp, or Tyr(OMe);

Xaa₁₂ is: Arg, Lys, Glu, hR, Orn, Lys (isopropyl), Aib, Cit, Ala, Leu, Gln, or Phe;

### Xaa₁₃ is: Leu, Phe, Glu, Ala, Aib, Ser, Cys, or K(CO(CH₂)₂SH);

Xaa₁₄ is: Arg, Leu, Lys, Ala, hR, Orn, Lys (isopropyl), Phe, Gln, Aib, or Cit;
Xaa₁₅ is: Lys, Ala, Arg, Glu, Leu, hR, Orn, Lys (isopropyl), Phe, Gln, Aib, K(Ac), Cit, or K(W);
Xaa₁₆ is: Gln, Lys, Glu, Ala, hR, Orn, Lys (isopropyl), Cit, Ser, Cys, or K(CO(CH₂)₂SH);

### Xaa₁₇ is: Val, Ala, Leu, Ile, Met, Nle, Lys, Aib, Ser, Cys, or K(CO(CH₂)₂SH);

### Xaa₁₈ is: Ala, Ser, or Cys;

### Xaa₁₉ is: Val, Ala, Glu, Phe, Gly, His, Ile, Lys, Leu, Met, Asn, Gln, Arg, Ser, Thr, Trp, Tyr, Cys, Asp, or K(CO(CH₂)₂SH);

Xaa₂₀ is: Lys, Gln, hR, Arg, Ser, His, Orn, Lys (isopropyl), Ala, Aib, Trp, Thr, Leu, Ile, Phe, Tyr, Val, K(Ac), Cit, or Cys;

### Xaa₂₁ is: Lys, His, Arg, Ala, Phe, Aib, Leu, Gln, Orn, hR, K(Ac), Cit, Ser, or Cys;

### Xaa₂₂ is: Tyr, Trp, Phe, Thr, Leu, Ile, Val, Tyr(OMe), Ala, Aib, Ser, or Cys;

### Xaa₂₃ is: Leu, Phe, Ile, Ala, Trp, Thr, Val, Aib, Ser, or Cys;

### Xaa₂₄ is: Gln, Glu, Asn, Ser, Cys, or K(CO(CH₂)₂SH);

### Xaa₂₅ is: Ser, Asp, Phe, Ile, Leu, Thr, Val, Trp, Gln, Asn, Tyr, Aib, Glu, Cys, or K(CO(CH₂)₂SH);

### Xaa₂₆ is: Ile, Leu, Thr, Val, Trp, Tyr, Phe, Ser, Cys, or K(CO(CH₂)₂SH);

Xaa₂₇ is: Lys, hR, Arg, Gln, Ala, Asp, Glu, Phe, Gly, His, Ile, Met, Asn, Ser, Thr, Val, Trp, Tyr, Lys (isopropyl), Cys, Leu, Orn, or dK;

### Xaa₂₈ is: Asn, Asp, Gln, Lys, Arg, Aib, Orn, hR, Cit, Pro, dK, Cys, or K(CO(CH₂)₂SH);

Xaa₂₉ is: Lys, Ser, Arg, Asn, hR, Ala, Asp, Glu, Phe, Gly, His, Ile, Leu, Met, Pro, Gln, Thr, Val, Trp, Tyr, Cys, Orn, Cit, Aib or is absent;
Xaa₃₀ is: Arg, Lys, Ile, Ala, Asp, Glu, Phe, Gly, His, Leu, Met, Asn, Pro, Gln, Ser, Thr, Val, Trp, Tyr, Cys, hR, Cit, Aib, Orn, or is absent;
Xaa₃₁ is: Tyr, His, Phe, Thr, Cys, or is absent;
Xaa₃₂ is: Ser, Cys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent

provided that if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the peptide agonist sequence,

and that the peptide agonist comprises at least one amino acid substitution selected from:

### Xaa₂ is: dA, Val, Gly, Leu, dS, or Aib;

### Xaa₄ is: Ile, Tyr, Phe, Val, Thr, Leu, Trp, dA, Aib, or NMeA;

### Xaa₅ is: Leu, Phe, Thr, Trp, Tyr, dV, or Aib;

### Xaa₈ is: Leu, Arg, or Tyr;

### Xaa₉ is: Glu, Ser, or Cys;

### Xaa₁₀ is: Trp;

Xaa₁₂ is: Ala, hR, Aib, Lys (isopropyl), Cit, Gln, or Phe;

### Xaa₁₃ is: Phe, Glu, Ala, Aib, Ser, Cys, or K(CO(CH₂)₂SH);

Xaa₁₄ is: Leu, Lys, Ala, hR, Orn, Lys (isopropyl), Phe, Gln, Aib, or Cit;
Xaa₁₅ is: Ala, Arg, Leu, hR, Orn, Lys (isopropyl), Phe, Gln, Aib, K(Ac), Cit, or K(W);
Xaa₁₆ is: Lys, (isopropyl), hR, Orn, Cit, Ser, Cys, or K(CO(CH₂)₂SH);

### Xaa₁₇ is: Lys, Aib, Ser, Cys, or K(CO(CH₂)₂SH);

### Xaa₁₈ is: Ser, or Cys;

### Xaa₁₉ is: K(CO(CH₂)₂SH);

### Xaa₂₀ is: Gln, hR, Arg, Ser, Orn, Lys(isopropyl), Ala, Aib, Trp, Thr, Leu, Ile, Phe, Tyr, Val, K(Ac), Cit, or Cys;

### Xaa₂₁ is: Arg, Ala, Phe, Aib, Leu, Gln, Orn, hR, K(Ac), Cit, Ser, or Cys;

### Xaa₂₂ is: Trp, Thr, Leu, Ile, Val, Tyr (OMe), Ala, Aib, Ser, or Cys;

### Xaa₂₃ is: Phe, Ile, Ala, Trp, Thr, Val, Aib, Ser, or Cys;

Xaa₂₄ is: Ser, Cys, or K(CO(CH₂)₂SH);

### Xaa₂₅ is: Phe, Ile, Leu, Val, Trp, Gln, Asn, Tyr, Aib, Glu, Cys, or K(CO(CH₂)₂SH);

### Xaa₂₆ is: Thr, Trp, Tyr, Phe, Ser, Cys, or K(CO(CH₂)₂SH);

### Xaa₂₇ is: hR, Orn, or dK;

### Xaa₂₈ is: Pro, Arg, Aib, Orn, hR, Cit, dK, Cys, or K(CO(CH₂)₂SH);

### Xaa₂₉ is: hR, Cys, Orn, Cit, or Aib;

### Xaa₃₀ is: hR, Cit, Aib, or Orn; and

### Xaa₃₁ is: His, or Phe.

According to a fourth aspect of the present invention, there is provided a cyclic VPAC2 receptor peptide agonist of the present invention for use as a medicament.

According to a fifth aspect of the present invention, there is provided the use of a cyclic VPAC2 receptor peptide agonist for the manufacture of a medicament for the treatment non-insulin-dependent diabetes.

According to a further aspect of the present invention, there is provided the use of a cyclic VPAC2 receptor peptide agonist for the manufacture of a medicament for the treatment of insulin-dependent diabetes.

According to yet a further aspect of the present invention, there is provided the use of a cyclic VPAC2 receptor peptide agonist for the manufacture of a medicament for the treatment of food intake suppression.

The VPAC2 receptor peptide agonists of the present invention, therefore, have the advantage that they have enhanced selectivity, potency and/or stability over known VPAC2 receptor peptide agonists. The addition of a C-terminal extension sequence surprisingly increased the VPAC2 receptor selectivity as well as increasing proteolytic stability. In particular, cyclic VPAC2 receptor peptide agonists have restricted conformational mobility compared to linear VPAC2 peptide receptor agonists of small/medium size and for this reason cyclic peptides have a smaller number of allowed conformations compared with linear peptides. Constraining the conformational flexibility of linear peptides by cyclisation enhances receptor-binding affinity, increases selectivity and improves proteolytic stability and bioavailability compared with linear peptides.

A “selective VPAC2 receptor peptide agonist” of the present invention is a peptide that selectively activates the VPAC2 receptor to induce insulin secretion. Preferably, the sequence for a selective VPAC2 receptor peptide agonist of the present invention has from about twenty-eight to about thirty-five naturally occurring and/or non-naturally occurring amino acids and may or may not additionally comprise a C-terminal extension. More preferably, the selective VPAC2 receptor peptide agonist has from twenty-eight to thirty-one naturally occurring and/or non-naturally occurring amino acids and may or may not additionally comprise a C-terminal extension.

A “selective cyclic VPAC2 receptor peptide agonist” or a “cyclic VPAC2 receptor peptide agonist” is a selective VPAC2 receptor peptide agonist cyclised by means of a covalent bond linking the side chains of two amino acids in the peptide chain. The covalent bond may, for example, be a lactam bridge or a disulfide bridge.

Selective cyclic VPAC2 receptor peptide agonists may have a C-terminal extension. The “C-terminal extension” of the present invention comprises a sequence having from one to thirteen naturally occurring or non-naturally occurring amino acids linked to the C-terminus of the sequence of Formula 1, 4, or 5 at the N-terminus of the C-terminal extension via a peptide bond.

As used herein, the term “linked to” with reference to the term C-terminal extension, includes the addition or attachment of amino acids or chemical groups directly to the C-terminus of the peptide of the Formula 1, 4, or 5.

Optionally, the selective cyclic VPAC2 receptor peptide agonist may also have an N-terminal modification. The term “N-terminal modification” as used herein includes the addition or attachment of amino acids or chemical groups directly to the N-terminus of a peptide and the formation of chemical groups, which incorporate the nitrogen at the N-terminus of a peptide.

The N-terminal modification may comprise the addition of one or more naturally occurring or non-naturally occurring amino acids to the VPAC2 receptor peptide agonist sequence, preferably there are not more than ten amino acids, with one amino acid being more preferred. Naturally occurring amino acids which may be added to the N-terminus include methionine and isoleucine. A modified amino acid added to the N-terminus may be D-histidine. Alternatively, the following amino acids may be added to the N-terminus: SEQ ID NO: 20 Ser-Trp-Cys-Glu-Pro-Gly-Trp-Cys-Arg, wherein the Arg is linked to the N-terminus of the peptide agonist. Preferably, any amino acids added to the N-terminus are linked to the N-terminus by a peptide bond.

The term “linked to” as used herein, with reference to the term N-terminal modification, includes the addition or attachment of amino acids or chemical groups directly to the N-terminus of the VPAC2 receptor agonist. The addition of the above N-terminal modifications may be achieved under normal coupling conditions for peptide bond formation.

The N-terminus of the peptide agonist may also be modified by the addition of an alkyl group (R), preferably a C₁-C₁₆ alkyl group, to form (R)NH—.

Alternatively, the N-terminus of the peptide agonist may be modified by the addition of a group of the formula —C(O)R¹ to form an amide of the formula R¹C(O)NH—. The addition of a group of the formula —C(O)R¹ may be achieved by reaction with an organic acid of the formula R¹COOH. Modification of the N-terminus of an amino acid sequence using acylation is demonstrated in the art (e.g. Gozes et al., J. Pharmacol Exp Ther, 273:161-167 (1995)). Addition of a group of the formula —C(O)R¹ may result in the formation of a urea group (see WO 01/23240, WO 2004/006839) or a carbamate group at the N-terminus. Also, the N-terminus may be modified by the addition of pyroglutamic acid or 6-aminohexanoic acid.

The N-terminus of the peptide agonist may be modified by the addition of a group of the formula —SO₂R⁵, to form a sulfonamide group at the N-terminus.

The N-terminus of the peptide agonist may also be modified by reacting with succinic anhydride to form a succinimide group at the N-terminus. The succinimide group incorporates the nitrogen at the N-terminus of the peptide.

The N-terminus may alternatively be modified by the addition of methionine sulfoxide, biotinyl-6-aminohexanoic acid, or —C(═NH)—NH₂. The addition of —C(═NH)—NH₂ is a guanidation modification, where the terminal NH₂ of the N-terminal amino acid becomes —NH—C(═NH)—NH₂.

Most of the sequences of the present invention, including the N-terminal modifications and the C-terminal extensions contain the standard single letter or three letter codes for the twenty naturally occurring amino acids. The other codes used are defined as follows:
Ac=Acetyl
C6=hexanoyl
d=the D isoform (nonnaturally occurring) of the respective amino acid, e.g., dA=D-alanine, dS=D-serine, dK=D-lysine
hR=homoarginine
_=position not occupied
Aib=amino isobutyric acid
CH₂=methylene
Met(O)=methionine sulfoxide
OMe=methoxy
Nle=Nor-leucine
NMe=N-methyl attached to the alpha amino group of an amino acid, e.g., NMeA=N-methyl alanine, NMeV=N-methyl valine
Orn=ornithine
Cit=citrulline
K(Ac)=ε-acetyl lysine
M=methionine
I=isoleucine
Dab=diaminobutyric acid
K(W)=ε-(L-tryptophyl)-lysine
K(CO(CH₂)₂SH)=ε-(3′-mercaptopropionyl)-lysine
Biotin-Acp=Biotinyl-6-aminohexanoic acid (6-aminocaproic acid)
[symbol] =a lactam or disulfide bridge

The term “VPAC2” is used to refer to and in conjunction with the particular receptor (Lutz, et al., FEBS Lett., 458: 197-203 (1999), Adamou, et al., Biochem. Biophys. Res. Commun., 209: 385-392 (1995)) that the agonists of the present invention activate. This term also is used to refer to and in conjunction with the agonists of the present invention.

VIP naturally occurs as a single sequence having 28 amino acids. However, PACAP exists as either a 38 amino acid peptide (PACAP-38) or as a 27 amino acid peptide (PACAP-27) with an amidated carboxyl (Miyata, et al., Biochem Biophys Res Commun, 170:643-648 (1990)). The sequences for VIP, PACAP-27, and PACAP-38 are as follows:

| | Seq. | |
|---|---|---|
| Peptide | ID # | Sequence |
| VIP | 21 | HSDAVFTDNYTRLRKQMAVKKYLNSILN |
| PACAP-27 | 22 | HSDGIFTDSYSRYRKQMAVKKYLAAVL-NH₂ |
| PACAP-38 | 23 | HSDGIFTDSYSRYRKQMAVKKYLAAVLGKRYQRVKN |
| | | K-NH₂ |

The term “naturally occurring amino acid” as used herein means the twenty amino acids coded for by the human genetic code (i.e. the twenty standard amino acids). These twenty amino acids are: Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine and Valine.

Examples of “non-naturally occurring amino acids” include both synthetic amino acids and those modified by the body. These include D-amino acids, arginine-like amino acids (e.g., homoarginine), and other amino acids having an extra methylene in the side chain (“homo” amino acids), and modified amino acids (e.g norleucine, lysine (isopropyl)—wherein the side chain amine of lysine is modified by an isopropyl group). Also included are amino acids such as ornithine and amino isobutyric acid.

“Selective” as used herein refers to a VPAC2 receptor peptide agonist with increased selectivity for the VPAC2 receptor compared to other known receptors. The degree of selectivity is determined by a ratio of VPAC2 receptor binding affinity to VPAC1 receptor binding affinity and by a ratio of VPAC2 receptor binding affinity to PAC1 receptor binding affinity. Preferably, the agonists of the present invention have a selectivity ratio where the affinity for the VPAC2 receptor is at least 50 times greater than, for the VPAC1 and/or for PAC1 receptors. More preferably, the affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the affinity is at least 1000 times greater for VPAC2 than for VPAC1 and/or for PAC1. Binding affinity is determined as described below in Example 4.

“Percent (%) sequence identity” as used herein is used to denote sequences which when aligned have similar (identical or conservatively replaced) amino acids in like positions or regions, where identical or conservatively replaced amino acids are those which do not alter the activity or function of the protein as compared to the starting protein. For example, two amino acid sequences with at least 85% identity to each other have at least 85% similar (identical or conservatively replaced residues) in a like position when aligned optimally allowing for up to 3 gaps, with the proviso that in respect of the gaps a total of not more than 15 amino acid residues is affected. Percent sequence identity may be calculated by determining the number of residues that differ between a peptide encompassed by the present invention and a reference peptide such as P57 (SEQ ID NO: 29), taking that number and dividing it by the number of amino acids in the reference peptide (e.g. 39 amino acids for P57), multiplying the result by 100, and subtracting that resulting number from 100. For example, a sequence having 39 amino acids with four amino acids that are different from P57 would have a percent (%) sequence identity of 90% (e.g. 100−(( 4/39)×100)). For a sequence that is longer than 39 amino acids, the number of residues that differ from the VIP sequence will include the additional amino acids over 39 for purposes of the aforementioned calculation. For example, a sequence having 41 amino acids, with four amino acids different from the 39 amino acids in the P57 sequence and with two additional amino acids at the carboxy terminus, which are not present in the P57 sequence, would have a total of six amino acids that differ from P57. Thus, this sequence would have a percent (%) sequence identity of 84% (e.g. 100−(( 6/39)×100)). The degree of sequence identity may be determined using methods well known in the art (see, for example, Wilbur, W. J. and Lipman, D. J., Proc. Natl. Acad. Sci. USA 80:726-730 (1983) and Myers E. and Miller W., Comput. Appl. Biosci. 4:11-17 (1988)). One program which may be used in determining the degree of similarity is the MegAlign Lipman-Pearson one pair method (using default parameters) which can be obtained from DNAstar Inc, 1128, Selfpark Street, Madison, Wis., 53715, USA as part of the Lasergene system. Another program, which may be used, is Clustal W. This is a multiple sequence alignment package developed by Thompson et al (Nucleic Acids Research, 22(22):4673-4680 (1994)) for DNA or protein sequences. This tool is useful for performing cross-species comparisons of related sequences and viewing sequence conservation. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen. Evolutionary relationships can be seen via viewing Cladograms or Phylograms.

The sequence for a selective cyclic VPAC2 receptor peptide agonist of the present invention is selective for the VPAC2 receptor and preferably has a sequence identity in the range of 50% to 60%, 50% to 55%, 55% to 60%, 60% to 70%, 60% to 65%, 65% to 70%, 70% to 80%, 70% to 75%, 75% to 80%, 80% to 90%, 80% to 85%, 85% to 90%, 90% to 97%, 90% to 95%, or 95% to 97% with P57 (SEQ ID NO: 29). Preferably, the sequence has a sequence identity of greater than 58% with P57 (SEQ ID NO: 29). More preferably, the sequence has greater than 76% sequence identity with P57 (SEQ ID NO: 29). Even more preferably, the sequence has greater than 84% sequence identity with P57 (SEQ ID NO: 29). Yet more preferably, the sequence has greater than 89% sequence identity with P57 (SEQ ID NO: 29).

The term “lactam bridge” as used herein means a covalent bond, in particular an amide bond, linking the side chain amino terminus of one amino acid in the peptide agonist to the side chain carboxy terminus of another amino acid in the peptide agonist. Preferably, the lactam bridge is formed by the covalent attachment of the side chain of a residue at Xaaₙ to the side chain of a residue at Xaaₙ₊₄, wherein n is 1 to 28. Also preferably, the lactam bridge is formed by the covalent attachment of the side chain amino terminus of a Lys, Orn, or Dab residue to the side chain carboxy terminus of an Asp or Glu residue.

The term “disulfide bridge” as used herein means a covalent bond linking a sulfur atom at the side chain terminus of one amino acid in the peptide agonist to a sulfur atom at the side chain terminus of another amino acid in the peptide agonist. Preferably, the disulfide bridge is formed by the covalent attachment of the side chain of a residue at Xaaₙ to the side chain of a residue at Xaaₙ₊₄, wherein n is 1 to 28. Also preferably, the disulfide bridge is formed by the covalent attachment of the side chain of a Cys or hC residue to the side chain of another Cys or hC residue.

The term “C₁-C₁₆ alkyl” as used herein means a monovalent saturated straight, branched or cyclic chain hydrocarbon radical having from 1 to 16 carbon atoms. Thus the term “C₁-C₁₆ alkyl” includes, for example, methyl, ethyl, n-propyl, isopropyl, n-butyl, isobutyl, sec-butyl, tert-butyl, n-heptyl, n-octyl, cyclopropyl, cyclobutyl, cyclopentyl and cyclohexyl. The C₁-C₁₆ alkyl group may be optionally substituted with one or more substituents.

The term “C₁-C₆ alkyl” as used herein means a monovalent saturated straight, branched or cyclic chain hydrocarbon radical having from 1 to 6 carbon atoms. Thus the term “C₁-C₆ alkyl” includes, for example, methyl, ethyl, n-propyl, isopropyl, n-butyl, isobutyl, sec-butyl, tert-butyl, cyclopropyl, cyclobutyl, cyclopentyl and cyclohexyl. The C₁-C₆ alkyl group may be optionally substituted with one or more substituents.

The term “C₂-C₆ alkenyl” as used herein means a monovalent straight, branched or cyclic chain hydrocarbon radical having at least one double bond and having from 2 to 6 carbon atoms. Thus the term “C₂-C₆ alkenyl” includes vinyl, prop-2-enyl, but-3-enyl, pent-4-enyl and isopropenyl. The C₂-C₆ alkenyl group may be optionally substituted with one or more substituents.

The term “C₂-C₆ alkynyl” as used herein means a monovalent straight or branched chain hydrocarbon radical having at least one triple bond and having from 2 to 6 carbon atoms. Thus the term “C₂-C₆ alkynyl” includes prop-2-ynyl, but-3-ynyl and pent-4-ynyl. The C₂-C₆ alkynyl may be optionally substituted with one or more substituents.

The term “halo” or “halogen” means fluorine, chlorine, bromine or iodine.

The term “aryl” when used alone or as part of a group is a 5 to 10 membered aromatic or heteroaromatic group including a phenyl group, a 5 or 6-membered monocyclic heteroaromatic group, each member of which may be optionally substituted with 1, 2, 3, 4 or 5 substituents (depending upon the number of available substitution positions), a naphtyl group or an 8-, 9- or 10-membered bicyclic heteroaromatic group each member of which may be optionally substituted with 1, 2, 3, 4, 5 or 6 substituents (depending on the number of available substitution positions). Within this definition of aryl, suitable substitutions include C₁-C₆ alkyl, C₂-C₆ alkenyl, C₂-C₆ alkynyl, amino, hydroxy, halogen, —SH and CF₃.

The term “aryl C₁-C₄ alkyl” as used herein means a C₁-C₄ alkyl group substituted with an aryl. Thus the term “aryl C₁-C₄ alkyl” includes benzyl, 1-phenylethyl (α-methylbenzyl), 2-phenylethyl, 1-naphthalenemethyl or 2-naphthalenemethyl.

The term “naphthyl” includes 1-naphthyl, and 2-naphthyl. 1-naphthyl is preferred.

The term “benzyl” as used herein means a monovalent unsubstituted phenyl radical linked to the point of substitution by a —CH₂— group.

The term “5- or 6-membered monocyclic heteroaromatic group” as used herein means a monocyclic aromatic group with a total of 5 or 6 atoms in the ring wherein from 1 to 4 of those atoms are each independently selected from N, O and S. Preferred groups have 1 or 2 atoms in the ring which are each independently selected from N, O and S. Examples of 5-membered monocyclic heteroaromatic groups include pyrrolyl (also called azolyl), furanyl, thienyl, pyrazolyl (also called 1H-pyrazolyl and 1,2-diazolyl), imidazolyl, oxazolyl (also called 1,3-oxazolyl), isoxazolyl (also called 1,2-oxazolyl), thiazolyl (also called 1,3-thiazolyl), isothiazolyl (also called 1,2-thiazolyl), triazolyl, oxadiazolyl, thiadiazolyl, tetrazolyl, oxatriazolyl and thiatriazolyl. Examples of 6-membered monocyclic heteroaromatic groups include pyridinyl, pyrimidyl, pyrazinyl, pyridazinyl and triazinyl.

The term “8-, 9- or 10-membered bicyclic heteroaromatic group” as used herein means a fused bicyclic aromatic group with a total of 8, 9 or 10 atoms in the ring system wherein from 1 to 4 of those atoms are each independently selected from N, O and S. Preferred groups have from 1 to 3 atoms in the ring system which are each independently selected from N, O and S. Suitable 8-membered bicyclic heteroaromatic groups include imidazo[2,1-b][1,3]thiazolyl, thieno[3,2-b]thienyl, thieno[2,3-d][1,3]thiazolyl and thieno[2,3-d]imidazolyl. Suitable 9-membered bicyclic heteroaromatic groups include indolyl, isoindolyl, benzofuranyl (also called benzo[b]furanyl), isobenzofuranyl (also called benzo[c]furanyl), benzothienyl (also called benzo[b]thienyl), isobenzothienyl (also called benzo[c]thienyl), indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazoyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl and imidazo[1,2-a]pyridine. Suitable 10-membered bicyclic heteroaromatic groups include quinolinyl, isoquinolinyl, cinnolinyl, quinazolinyl, quinoxalinyl, 1,5-naphthyridyl, 1,6-naphthyridyl, 1,7-naphthyridyl and 1,8-naphthyridyl.

The term “C₁-C₆ alkoxy” as used herein means a monovalent unsubstituted saturated straight-chain or branched-chain hydrocarbon radical having from 1 to 6 carbon atoms linked to the point of substitution by a divalent O radical. Thus the term “C₁-C₆ alkoxy” includes, for example, methoxy, ethoxy, n-propoxy, isopropoxy, n-butoxy, isobutoxy, sec-butoxy and tert-butoxy. The C₁-C₆ alkoxy group may be optionally substituted with one or more substituents.

“Insulinotropic activity” refers to the ability to stimulate insulin secretion in response to elevated glucose levels, thereby causing glucose uptake by cells and decreased plasma glucose levels. Insulinotropic activity can be assessed by methods known in the art, including using experiments that measure VPAC2 receptor binding activity or receptor activation (e.g. insulin secretion by insulinoma cell lines or islets, intravenous glucose tolerance test (IVGTT), intraperitoneal glucose tolerance test (IPGTT), and oral glucose tolerance test (OGTT)). Insulinotropic activity is routinely measured in humans by measuring insulin levels or C-peptide levels. Selective cyclic VPAC2 receptor peptide agonists of the present invention have insulinotropic activity.

“In vitro potency” as used herein is the measure of the ability of a peptide to activate the VPAC2 receptor in a cell-based assay. In vitro potency is expressed as the “EC₅₀” which is the effective concentration of compound that results in a 50% of maximum increase in activity in a single dose-response experiment. For the purposes of the present invention, in vitro potency is determined using two different assays: DiscoveRx and Alpha Screen. See Example 3 for further details of these assays. Whilst these assays are performed in different ways, the results demonstrate a general correlation between the two assays.

According to a preferred embodiment of the present invention, there is provided a cyclic VPAC2 receptor peptide agonist comprising an amino acid sequence of Formula 1 (SEQ ID NO:1). Formula 4 (SEQ ID NO: 4), or Formula 5 (SEQ ID NO: 5), wherein the peptide agonist is cyclised by means of a lactam bridge and the lactam bridge is formed by the covalent attachment of the side chain of the residue at Xaaₙ and the side chain of the residue at Xaaₙ₊₄. In this embodiment, it is preferred that n is 21. It is also preferred that the lactam bridge is formed by the covalent attachment of the side chain of a Lys, Orn, or Dab residue to the side chain of an Asp or Glu residue.

According to another preferred embodiment of the present invention, there is provided a cyclic VPAC2 receptor peptide agonist comprising an amino acid sequence of Formula 1 (SEQ ID NO: 1), Formula 4 (SEQ ID NO: 4), or Formula 5 (SEQ ID NO: 5), wherein the peptide agonist is cyclised by means of a disulfide bridge and the disulfide bridge is formed by the covalent attachment of the side chain of the residue at Xaaₙ and the side chain of the residue at Xaaₙ₊₄. In this embodiment, it is preferred that n is 12 or 21. It is also preferred that the disulfide bridge is formed by the covalent attachment of the side chain of a Cys or hC residue to the side chain of another Cys or hC residue.

In one preferred embodiment of the present invention, there is provided a cyclic VPAC2 receptor peptide agonist comprising an amino acid sequence of Formula 1 (SEQ ID NO: 1), Formula 4 (SEQ ID NO: 4), or Formula 5 (SEQ ID NO: 5), wherein Xaa₁₂ is Lys, Orn, or hR, Xaa₁₃ is Leu, or Aib, Xaa₁₅ is Lys, Aib, or Orn, Xaa₂₀ is Lys, or Orn, Xaa₂₇ is Lys, Orn, or hR, Xaa₂₈ is Lys, Orn, Aib, Gln, hR, or Pro, Xaa₂₉ is Orn, Lys, hR, or absent, and Xaa₃₀ and all subsequent residues are absent, and a C-terminal extension comprising an amino acid sequence of Formula 6 (SEQ ID NO: 6). It is more preferred that the C-terminal extension in this embodiment is selected from:

| | |
|---|---|
| SEQ ID NO: 10 | GGPSSGAPPPS |
| SEQ ID NO: 11 | GGPSSGAPPPS-NH₂ |
| SEQ ID NO: 12 | GGPSSGAPPPC |
| SEQ ID NO: 13 | GGPSSGAPPPC-NH₂ |
| SEQ ID NO: 14 | GRPSSGAPPPS |
| SEQ ID NO: 15 | GRPSSGAPPPS-NH₂ |

In another preferred embodiment of the present invention, there is provided a cyclic VPAC2 receptor peptide agonist comprising an amino acid sequence of Formula 1 (SEQ ID NO: 1), Formula 4 (SEQ ID NO: 4), or Formula 5 (SEQ ID NO: 5), wherein Xaa₁₂ is Lys, Orn, or hR, Xaa₁₃ is Leu, or Aib, Xaa₁₅ is Lys, Aib, or Orn, Xaa₂₀ is Lys, or Orn, Xaa₂₇ is Lys, Orn, or hR, Xaa₂₈ is Lys, Orn, Aib, Gln, hR, or Pro, Xaa₂₉ is Orn, Lys, hR, or absent, and Xaa₃₀ and all subsequent residues are absent, and a C-terminal extension comprising an amino acid sequence of Formula 7 (SEQ ID NO: 7). It is more preferred that the C-terminal extension in this embodiment is selected from:

| | |
|---|---|
| SEQ ID NO: 16 | SRTSPPP |
| SEQ ID NO: 17 | SRTSPPP-NH₂ |
| SEQ ID NO: 18 | SSTSPRPPSS |
| SEQ ID NO: 19 | SSTSPRPPSS-NH₂ |

In another preferred embodiment of the present invention, there is provided a cyclic VPAC2 receptor peptide agonist comprising an amino acid sequence of Formula 5 (SEQ ID NO: 5) and a C-terminal extension comprising an amino acid sequence of Formula 6 (SEQ ID NO: 6). It is more preferred that the C-terminal extension in this embodiment is selected from:

| | |
|---|---|
| SEQ ID NO: 10 | GGPSSGAPPPS |
| SEQ ID NO: 11 | GGPSSGAPPPS-NH₂ |
| SEQ ID NO: 12 | GGPSSGAPPPC |
| SEQ ID NO: 13 | GGPSSGAPPPC-NH₂ |
| SEQ ID NO: 14 | GRPSSGAPPPS |
| SEQ ID NO: 15 | GRPSSGAPPPS-NH₂ |

In yet another preferred embodiment of the present invention, there is provided a cyclic VPAC2 receptor peptide agonist comprising an amino acid sequence of Formula 5 (SEQ ID NO: 5) and a C-terminal extension comprising an amino acid sequence of Formula 7 (SEQ ID NO:7). It is more preferred that the C-terminal extension in this embodiment is selected from:

| | |
|---|---|
| SEQ ID NO: 16 | SRTSPPP |
| SEQ ID NO: 17 | SRTSPPP-NH₂ |
| SEQ ID NO: 18 | SSTSPRPPSS |
| SEQ ID NO: 19 | SSTSPRPPSS-NH₂ |

In the above preferred embodiments of the present invention, it is especially preferred that the VPAC2 receptor peptide agonist further comprises an N-terminal modification, wherein the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂ and even more preferably, is the addition of acetyl, hexanoyl, cyclohexanoyl, or propionyl

According to a preferred embodiment of the present invention, there is provided a cyclic VPAC2 receptor peptide agonist comprising an amino acid sequence of Formula 5 (SEQ ID NO: 5), and a C-terminal extension selected from: GGPSSGAPPPS (SEQ ID NO: 10), GGPSSGAPPPS—NH₂ (SEQ ID NO: 11), GGPSSGAPPPC (SEQ ID NO: 12), GGPSSGAPPPC—NH₂ (SEQ ID NO: 13), GRPSSGAPPPS (SEQ ID NO: 14), and GRPSSGAPPPS—NH₂ (SEQ ID NO: 15), wherein the peptide agonist is cyclised by means of a lactam bridge linking the side chain of a Lys, Orn or Dab residue at Xaa₂₁ to the side chain of an Asp or Glu residue at Xaa₂₅ and wherein the VPAC2 receptor peptide agonist further comprises a N-terminal modification which modification is the addition of hexanoyl, acetyl, cyclohexanoyl or propionyl.

According to another preferred embodiment of the present invention, there is provided a cyclic VPAC2 receptor peptide agonist comprising an amino acid sequence of Formula 5 (SEQ ID NO: 5), wherein Xaa₁₂ is Lys, Orn, or hR, Xaa₁₃ is Leu, or Aib, Xaa₁₅ is Lys, Aib, or Orn, Xaa₂₀ is Lys, or Orn, Xaa₂₇ is Lys, Orn, or hR, Xaa₂₈ is Lys, Orn, Aib, Gln, hR, or Pro, Xaa₂₉ is Orn, Lys, hR, or absent, and Xaa₃₀ and all subsequent residues are absent, and a C-terminal extension selected from: GGPSSGAPPPS (SEQ ID NO: 10), GGPSSGAPPPS—NH₂ (SEQ ID NO: 11), GGPSSGAPPPC (SEQ ID NO: 12), GGPSSGAPPPC—NH₂ (SEQ ID NO: 13), GRPSSGAPPPS (SEQ ID NO: 14), and GRPSSGAPPPS—NH₂ (SEQ ID NO: 15), wherein the peptide agonist is cyclised by means of a lactam bridge linking the side chain of a Lys, Orn or Dab residue at Xaa₂₁ to the side chain of an Asp or Glu residue at Xaa₂₅ and wherein the VPAC2 receptor peptide agonist further comprises a N-terminal modification which modification is the addition of hexanoyl, acetyl, cyclohexanoyl or propionyl.

According to yet another preferred embodiment of the present invention, there is provided a cyclic VPAC2 receptor peptide agonist comprising an amino acid sequence of Formula 5 (SEQ ID NO: 5), wherein Xaa₁₂ is Lys, Orn, or hR, Xaa₁₃ is Leu, or Aib, Xaa₁₅ is Lys, Aib, or Orn, Xaa₂₀ is Lys, or Orn, Xaa₂₇ is Lys, Orn, or hR, Xaa₂₈ is Lys, Orn, Aib, Gln, hR, or Pro, Xaa₂₉ is Orn, Lys, hR, or absent, and Xaa₃₀ and all subsequent residues are absent, and a C-terminal extension selected from: GGPSSGAPPPS (SEQ ID NO: 10), GGPSSGAPPPS—NH₂ (SEQ ID NO: 11), GGPSSGAPPPC (SEQ ID NO: 12), GGPSSGAPPPC—NH₂ (SEQ ID NO: 13), GRPSSGAPPPS (SEQ ID NO: 14), and GRPSSGAPPPS—NH₂ (SEQ ID NO: 15), wherein the peptide agonist is cyclised by means of a lactam bridge linking the side chain of a Lys residue at Xaa₂₁ to the side chain of an Asp residue at Xaa₂₅ and wherein the VPAC2 receptor peptide agonist further comprises a N-terminal modification which modification is the addition of hexanoyl, acetyl, cyclohexanoyl or propionyl.

The region of wild-type VIP from aspartic acid at position 8 to isoleucine at position 26 has an alpha-helix structure. Increasing the helical content of a peptide enhances potency and selectivity whilst at the same time improving protection from enzymatic degradation. The use of a C-terminal extension, such as an exendin-4 extension, may enhance the helicity of the peptide. In addition, the introduction of a covalent bond, for example a lactam bridge, linking the side chains of two amino acids on the surface of the helix, also enhances the helicity of the peptide.

The present invention also encompasses the discovery that specific amino acids added to the C-terminus of a peptide sequence for a VPAC2 receptor peptide agonist provide features that may protect the peptide as well as may enhance activity, selectivity, and/or potency. For example, these C-terminal extensions may stabilize the helical structure of the peptide and stabilise sites located near to the C-terminus, which are prone to enzymatic cleavage. Furthermore, many of the C-terminally extended peptides disclosed herein may be more selective for the VPAC2 receptor and can be more potent than VIP, PACAP, and other known VPAC2 receptor peptide agonists. An example of a preferred C-terminal extension is the extension peptide of exendin-4 as the C-capping sequence. Exendin-4 is found in the salivary excretions from the Gila Monster, Heloderma Suspectum, (Eng et al., J. Biol. Chem., 267(11):7402-7405 (1992)). Another example of preferred C-terminal extension is the C-terminal sequence of helodermin. Helodermin is also found in the salivary excretions of the Gila Monster.

It has furthermore been discovered that modification of the N-terminus of the VPAC2 receptor peptide agonist may enhance potency and/or provide stability against DPP-IV cleavage.

VIP and some known VPAC2 receptor peptide agonists are susceptible to cleavage by various enzymes and, thus, have a short in vivo half-life. Various enzymatic cleavage sites in the VPAC2 receptor peptide agonists are discussed below. The cleavage sites are discussed relative to the amino acid positions in VIP (SEQ ID NO: 21), and are applicable to the sequences noted herein.

Cleavage of the peptide agonist by the enzyme dipeptidyl-peptidase-IV (DPP-IV) occurs between position 2 (serine in VIP) and position 3 (aspartic acid in VIP). The addition of a N-terminal modification and/or various substitutions at position 2 may improve stability against DPP-IV cleavage. Examples of N-terminal modifications that may improve stability against DPP-IV inactivation include the addition of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid and —C(═NH)—NH₂. Preferably, the N-terminal modification is the addition of acetyl, hexanoyl, cyclohexanoyl or propionyl.

There are chymotrypsin cleavage sites in wild-type VIP between the amino acids 10 and 11 (tyrosine and threonine) and those at 22 and 23 (tyrosine and leucine). Substituting Tyr(OMe) for tyrosine may increase stability at the 10-11 site. A lactam bridge, for example, linking the side chains of the amino acids at positions 21 and 25 protects the 22-23 site from cleavage.

There is a trypsin cleavage site between the amino acids at positions 12 and 13 of wild-type VIP. Certain amino acids render the peptide less susceptible to cleavage at this site, for example, ornithine and homoarginine at position 12 and amino isobutyric acid at position 13. These amino acids are, therefore, preferred at these positions.

In wild-type VIP, and in numerous VPAC2 receptor peptide agonists known in the art, there are cleavage sites between the basic amino acids at positions 14 and 15 and between those at positions 20 and 21. The selective cyclic VPAC2 receptor peptide agonists of the present invention generally have improved proteolytic stability in-vivo due to substitutions at these sites. The preferred substitutions at these sites are those which render the peptide less susceptible to cleavage by trypsin-like enzymes, including trypsin. For example, glutamine, amino isobutyric acid, homoarginine, ornithine, citrulline, lysine, alanine, and leucine are preferred at position 14, amino isobutyric acid and ornithine are preferred at position 15 and ornithine is preferred at position 20.

The bond between the amino acids at positions 25 and 26 of wild-type VIP is susceptible to enzymatic cleavage. This cleavage site may be completely or partially eliminated through substitution of the amino acid at position 25 and/or the amino acid at position 26.

The region of the VPAC2 receptor peptide agonist encompassing the amino acids at positions 27, 28, 29, 30 and 31 is also susceptible to enzyme cleavage. The addition of a C-terminal extension may render the peptide agonist more stable against neuroendopeptidase (NEP), it may also increase selectivity for the VPAC2 receptor. This region may also be attacked by trypsin-like enzymes. If this occurs, the peptide agonist may lose its C-terminal extension with the additional carboxypeptidase activity leading to an inactive form of the peptide. Preferred substitutions which may increase resistance to cleavage in this region include ornithine, homoarginine or lysine at position 27, lysine, ornithine, amino isobutyric acid, glutamine, homoarginine or proline at position 28 and ornithine, lysine, or homoarginine at position 29. Alternatively, Xaa₂₉ may be absent. Omitting the residues at position 30 onwards in Formula 1, 4, or 5, such that the C-terminal extension is bonded directly to the residue at position 28 or 29, may also increase resistance to enzymatic cleavage.

In addition to selective VPAC2 receptor peptide agonists with resistance to cleavage by various peptidases, the selective cyclic VPAC2 peptide receptor agonists of the present invention may also encompass peptides with enhanced selectivity for the VPAC2 receptor, increased potency, and/or increased stability compared with some peptides known in the art. Examples of amino acid positions that may affect such properties include positions: 3, 8, 12, 14, 15, 16, 20, 21, 25, 26, 27, 28, and 29 of Formula 1, 4 and 5. Preferred substitutions at these position include those in Formula 5.

The increased potency and selectivity for various cyclic VPAC2 receptor peptide agonists of the present invention is demonstrated in Examples 3 and 4. For example, Table 1 in Example 3 provides a list of selective cyclic VPAC2 receptor peptide agonists and their corresponding in vitro potency results. Preferably, the selective VPAC2 receptor peptide agonists of the present invention have an EC₅₀ value less than 2 nM. More preferably, the EC₅₀ value is less than 1 nM. Even more preferably, the EC₅₀ value is less than 0.5 nM. Still more preferably, the EC₅₀ value is less than 0.1 nM.

Table 2 in Example 4 provides a list of cyclic VPAC2 receptor peptide agonists and their corresponding selectivity results for human VPAC2, VPAC1, and PAC1. See Example 4 for further details of these assays. These results are provided as a ratio of VPAC2 binding affinity to VPAC1 binding affinity and as a ratio of VPAC2 binding affinity to PAC1 binding affinity. Preferably, the agonists of the present invention have a selectivity ratio where the affinity for the VPAC2 receptor is at least 50 times greater than for the VPAC1 and/or for PAC1 receptors. More preferably, this affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the ratio is at least 1000 times greater for VPAC2 than for VPAC1 and/or for PAC1.

As used herein, “selective cyclic VPAC2 receptor peptide agonists” also include pharmaceutically acceptable salts of the compounds described herein. A selective cyclic VPAC2 receptor peptide agonist of this invention can possess a sufficiently acidic, a sufficiently basic, or both functional groups, and accordingly react with any of a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, trifluoroacetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The selective cyclic VPAC2 receptor peptide agonists of the present invention can be administered parenterally. Parenteral administration can include, for example, systemic administration, such as by intramuscular, intravenous, subcutaneous, intradermal, or intraperitoneal injection. These agonists can be administered to the subject in conjunction with an acceptable pharmaceutical carrier, diluent, or excipient as part of a pharmaceutical composition for treating NIDDM, or the disorders discussed below. The pharmaceutical composition can be a solution or, if administered parenterally, a suspension of the VPAC2 receptor peptide agonist or a suspension of the VPAC2 receptor peptide agonist complexed with a divalent metal cation such as zinc. Suitable pharmaceutical carriers may contain inert ingredients which do not interact with the peptide or peptide derivative. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include lactose, dextrose, sucrose, trehalose, sorbitol, and mannitol.

Standard pharmaceutical formulation techniques may be employed such as those described in Remington's Pharmaceutical Sciences, Mack Publishing Company, Easton, Pa. The selective cyclic VPAC2 receptor peptide agonists of the present invention may be formulated for administration through the buccal, topical, oral, transdermal, nasal, or pulmonary route.

The cyclic VPAC2 receptor peptide agonists of the invention may be formulated for administration such that blood plasma levels are maintained in the efficacious range for extended time periods. The main barrier to effective oral peptide drug delivery is poor bioavailability due to degradation of peptides by acids and enzymes, poor absorption through epithelial membranes, and transition of peptides to an insoluble form after exposure to the acidic pH environment in the digestive tract. Oral delivery systems for peptides such as those encompassed by the present invention are known in the art. For example, cyclic VPAC2 receptor peptide agonists can be encapsulated using microspheres and then delivered orally. For example, cyclic VPAC2 receptor peptide agonists can be encapsulated into microspheres composed of a commercially available, biocompatible, biodegradable polymer, poly(lactide-co-glycolide)-COOH and olive oil as a filler (see Joseph, et al. Diabetologia 43:1319-1328 (2000)). Other types of microsphere technology is also available commercially such as Medisorb® and Prolease® biodegradable polymers from Allermes. Medisorb® polymers can be produced with any of the lactide isomers. Lactide:glycolide ratios can be varied between 0:100 and 100:0 allowing for a broad range of polymer properties. This allows for the design of delivery systems and implantable devices with resorption times ranging from weeks to months. Emisphere has also published numerous articles discussing oral delivery technology for peptides and proteins. For example, see WO 95/28838 by Leone-bay et al. which discloses specific carriers comprised of modified amino acids to facilitate absorption.

The selective cyclic VPAC2 receptor peptide agonists described herein can be used to treat subjects with a wide variety of diseases and conditions. Agonists encompassed by the present invention exert their biological effects by acting at a receptor referred to as the VPAC2 receptor. Subjects with diseases and/or conditions that respond favourably to VPAC2 receptor stimulation or to the administration of VPAC2 receptor peptide agonists can therefore be treated with the VPAC2 agonists of the present invention. These subjects are said to “be in need of treatment with VPAC2 agonists” or “in need of VPAC2 receptor stimulation”.

The selective cyclic VPAC2 receptor peptide agonists of the present invention may be employed to treat diabetes, including both type 1 and type 2 diabetes (non-insulin dependent diabetes mellitus or NIDDM). Also included are subjects requiring prophylactic treatment with a VPAC2 receptor agonist, e.g., subjects at risk for developing NIDDM. Such treatment may also delay the onset of diabetes and diabetic complications. Additional subjects include those with impaired glucose tolerance or impaired fasting glucose, subjects whose body weight is about 25% above normal body weight for the subject's height and body build, subject's having one or more parents with NIDDM, subjects who have had gestational diabetes, and subjects with metabolic disorders such as those resulting from decreased endogenous insulin secretion. The selective cyclic VPAC2 receptor peptide agonists may be used to prevent subjects with impaired glucose tolerance from proceeding to develop type 2 diabetes, prevent pancreatic β-cell deterioration, induce β-cell proliferation, improve β-cell function, activate dormant β-cells, differentiate cells into β-cells, stimulate β-cell replication, and inhibit β-cell apoptosis. Other diseases and conditions that may be treated or prevented using compounds of the invention in methods of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); impaired glucose tolerance (IGT) (Expert Committee on Classification of Diabetes Mellitus, Diabetes Care 22 (Supp. 1):S5, 1999); impaired fasting glucose (IFG) (Charles, et al., Diabetes 40:796, 1991); gestational diabetes (Metzger, Diabetes, 40:197, 1991); metabolic syndrome X, dyslipidemia, hyperglycemia, hyperinsulinemia, hypertriglyceridemia, and insulin resistance.

The selective cyclic VPAC2 receptor peptide agonists of the invention may also be used in methods of the invention to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenyloin, thyroid hormone, β-adrenergic agents, α-interferon and drugs used to treat HIV infection.

The selective cyclic VPAC2 receptor peptide agonists of the present invention may be effective in the suppression of food intake and the treatment of obesity.

The selective cyclic VPAC2 receptor peptide agonists of the present invention may also be effective in the prevention or treatment of such disorders as atherosclerotic disease, hyperlipidemia, hypercholesteremia, low HDL levels, hypertension, primary pulmonary hypertension, cardiovascular disease (including atherosclerosis, coronary heart disease, coronary artery disease, and hypertension), cerebrovascular disease and peripheral vessel disease; and for the treatment of lupus, polycystic ovary syndrome, carcinogenesis, and hyperplasia, asthma, male and female reproduction problems, sexual disorders, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, rheumatoid arthritis, and septic shock.

The selective cyclic VPAC2 receptor peptide agonists of the present invention may also be useful for treating physiological disorders related to, for example, cell differentiation to produce lipid accumulating cells, regulation of insulin sensitivity and blood glucose levels, which are involved in, for example, abnormal pancreatic β-cell function, insulin secreting tumors and/or autoimmune hypoglycemia due to autoantibodies to insulin, autoantibodies to the insulin receptor, or autoantibodies that are stimulatory to pancreatic β-cells, macrophage differentiation which leads to the formation of atherosclerotic plaques, inflammatory response, carcinogenesis, hyperplasia, adipocyte gene expression, adipocyte differentiation, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

In addition, the selective VPAC2 receptor peptide agonists of the invention may be used for treatment of asthma (Bolin, et al., Biopolymer 37:57-66 (1995); U.S. Pat. No. 5,677,419; showing that polypeptide R3PO is active in relaxing guinea pig tracheal smooth muscle); hypotension induction (VIP induces hypotension, tachycardia, and facial flushing in asthmatic patients (Morice, et al., Peptides 7:279-280 (1986); Morice, et al., Lancet 2:1225-1227 (1983)); male reproduction problems (Siow, et al., Arch. Androl. 43(1):67-71 (1999)); as an anti-apoptosis/neuroprotective agent (Brenneman, et al., Ann. N.Y. Acad. Sci. 865:207-12 (1998)); cardioprotection during ischemic events (Kalfin, et al., J. Pharmacol. Exp. Ther. 1268(2):952-8 (1994); Das, et al., Ann. N.Y. Acad. Sci. 865:297-308 (1998)), manipulation of the circadian clock and its associated disorders (Hamar, et al., Cell 109:497-508 (2002); Shen, et al., Proc. Natl. Acad. Sci. 97:11575-80, (2000)), and as an anti-ulcer agent (Tuncel, et al., Ann. N.Y. Acad. Sci. 865:309-22, (1998)).

An “effective amount” of a selective cyclic VPAC2 receptor peptide agonist is the quantity that results in a desired therapeutic and/or prophylactic effect without causing unacceptable side effects when administered to a subject in need of VPAC2 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a cyclic VPAC2 agonist for the treatment of NIDDM is the quantity that would result in greater control of blood glucose concentration than in the absence of treatment, thereby resulting in a delay in the onset of diabetic complications such as retinopathy, neuropathy, or kidney disease. An “effective amount” of a selective cyclic VPAC2 receptor peptide agonist for the prevention of NIDDM is the quantity that would delay, compared with the absence of treatment, the onset of elevated blood glucose levels that require treatment with anti-hypoglycemic drugs such as sulfonylureas, thiazolidinediones, insulin, and/or bisguanidines.

An “effective amount” of the selective cyclic VPAC2 receptor peptide agonist administered to a subject will also depend on the type and severity of the disease and on the characteristics of the subject, such as general health, age, sex, body weight and tolerance to drugs. The dose of selective cyclic VPAC2 peptide receptor agonist effective to normalize a patient's blood glucose will depend on a number of factors, among which are included, without limitation, the subject's sex, weight and age, the severity of inability to regulate blood glucose, the route of administration and bioavailability, the pharmacokinetic profile of the peptide, the potency, and the formulation.

A typical dose range for the selective cyclic VPAC2 receptor peptide agonists of the present invention will range from about 1 μg per day to about 5000 μg per day. Preferably, the dose ranges from about 1 μg per day to about 2500 μg per day, more preferably from about 1 μg per day to about 1000 μg per day. Even more preferably, the dose ranges from about 5 μg per day to about 100 μg per day. A further preferred dose range is from about 10 μg per day to about 50 μg per day. Most preferably, the dose is about 20 μg per day.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

The selective VPAC2 receptor peptide agonists of the present invention can be prepared by using standard methods of solid-phase peptide synthesis techniques. Peptide synthesizers are commercially available from, for example, Applied Biosystems, ABI 433A Peptide Synthesizer. Reagents for solid phase synthesis are commercially available, for example, from Glycopep (Chicago, Ill.). Solid phase peptide synthesizers can be used according to manufacturers instructions for blocking interfering groups, protecting the amino acid to be reacted, coupling, decoupling, and capping of unreacted amino acids.

Typically, an α-N-protected amino acid and the N-terminal amino acid on the growing peptide chain on a resin is coupled at room temperature in an inert solvent such as dimethylformamide, N-methylpyrrolidone or methylene chloride in the presence of coupling agents such as dicyclohexylcarbodiimide and 1-hydroxybenzotriazole and a base such as diisopropylethylamine. The α-N-protecting group is removed from the resulting peptide resin using a reagent such as trifluoroacetic acid or piperidine, and the coupling reaction repeated with the next desired N-protected amino acid to be added to the peptide chain. Suitable amine protecting groups are well known in the art and are described, for example, in Green and Wuts, “Protecting Groups in Organic Synthesis”, John Wiley and Sons, 1991. Examples include t-butyloxycarbonyl (tBoc) and fluorenylmethoxycarbonyl (Fmoc).

The selective VPAC2 receptor peptide agonists may also be synthesized using standard automated solid-phase synthesis protocols using t-butoxycarbonyl- or fluorenylmethoxycarbonyl-alpha-amino acids with appropriate side-chain protection. After completion of synthesis, peptides are cleaved from the solid-phase support with simultaneous side-chain deprotection using standard hydrogen fluoride methods or trifluoroacetic acid (TFA). Crude peptides are then further purified using Reversed-Phase Chromatography on Vydac C18 columns using acetonitrile gradients in 0.1% trifluoroacetic acid (TFA). To remove acetonitrile, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

The peptide agonists of the present invention may also be made by recombinant methods known in the art using both eukaryotic and prokaryotic cellular hosts.

The cyclisation of the VPAC2 receptor peptide agonists can be carried out in solution or on a solid support. Cyclisation on a solid support can be performed immediately following solid phase synthesis of the peptide. This involves the selective or orthogonal protection of the amino acids which will be covalently linked in the cyclisation.

Various preferred features and embodiments of the present invention will now be described with reference to the following non-limiting examples.

### EXAMPLE 1

### Preparation of the Selective Cyclic VPAC2 Receptor Peptide Agonists by Solid Phase t-Boc Chemistry

Approximately 0.5-0.6 grams (0.35-0.45 mmole) Boc Ser(Bzl)-PAM resin is placed in a standard 60 mL reaction vessel. Double couplings are run on an Applied Biosystems ABI433A peptide synthesizer. The following side-chain protected amino acids (2 mmole cartridges of Boc amino acids) are obtained from Midwest Biotech (Fishers, Ind.) and are used in the synthesis:

Arg-tosyl (Tos), Asp-cyclohexyl ester(OcHx), Asp-9-fluorenylmethyl (Fm), Cys-p-methylbenzyl (p-MeBzl), Glu-cyclohexyl ester (OcHx), His-benzyloxymethyl(Bom), Lys-2-chlorobenzyloxycarbonyl (2Cl-Z), Lys-9-fluorenylmethoxycarbonyl (Fmoc), Orn-2-chlorobenzyloxycarbonyl (2Cl-Z), Ser-O-benzyl ether (OBzl), Thr-O-benzyl ether (OBzl), Tyr-2-bromobenzyloxycarbonyl (2Br-Z), Boc-Ser(OBzl) PAM resin, and MBHA resin. Trifluoroacetic acid (TFA), di-isopropylethylamine (DIEA), 1.0 M hydroxybenzotriazole (HOBt) in NMP and 1.0 M dicyclohexylcarbodiimide (DCC) in NMP are purchased from PE-Applied Biosystems (Foster City, Calif.). Dimethylformamide (DMF-Burdick and Jackson) and dichloromethane (DCM-Mallinkrodt) is purchased from Mays Chemical Co. (Indianapolis, Ind.). Benzotriazole-1-yl-oxy-tris-(dimethylamino)-phosphoniumhexafluorophosphate (BOP) is obtained from NovaBiochem (San Diego, Calif.).

Cyclic VPAC2 receptor peptide agonists with a lactam bridge linking a lysine residue and an aspartic acid residue are prepared by selectively protecting the side chains of these residues with Fmoc and Fm, respectively. All other amino acids used in the synthesis are standard benzyl side-chain protected Boc-amino acids.

Standard double couplings are run using either symmetric anhydride or HOBt esters, both formed using DCC. At the completion of the syntheses, the N-terminal Boc group is removed and the peptidyl resins are capped with an organic acid such as hexanoic acid using diisopropylcarbodiimide (DIC) in DMF. The resin is then treated with 20% piperidine in DMF for 20 min. The Fmoc and Fm protecting groups are selectively removed and the cyclisation is carried out by activating the aspartic acid carboxyl group with BOP in the presence of DIEA. The reaction is allowed to proceed for 24 hours and monitored by ninhydrin test. After washing with DCM, the resins are transferred to a TEFLON reaction vessel and are dried in vacuo.

Cleavages are done by attaching the reaction vessels to a HF (hydrofluoric acid) apparatus (Penninsula Laboratories). 1 mL m-cresol per gram/resin is added and 10 mL HF (purchased from AGA, Indianapolis, Ind.) is condensed into the pre-cooled vessel. 1 mL DMS per gram resin is added when methionine is present. The reactions are stirred one hour in an ice bath. The HF is removed in vacuo. The residues are suspended in ethyl ether. The solids are filtered and are washed with ether. Each peptide is extracted into aqueous acetic acid and either is freeze dried or is loaded directly onto a reverse-phase column.

Purifications are run on a 2.2×25 cm VYDAC C18 column in buffer A (0.1% Trifluoroacteic acid in water, B: 0.1% TFA in acetonitrile). A gradient of 20% to 90% B is run on an HPLC (Waters) over 120 minutes at 10 mL/minute while monitoring the UV at 280 nm (4.0 A) and collecting one minute fractions. Appropriate fractions are combined, frozen and lyophilized. Dried products are analyzed by HPLC (0.46×15 cm METASIL AQ C18) and MALDI mass spectrometry.

### EXAMPLE 2

### Preparation of the Selective VPAC2 Receptor Cyclic Peptide Agonists by Solid Phase Fmoc Chemistry

Approximately 114 mg (50 mmole) Fmoc-Ser(tBu) WANG resin (purchased from GlycoPep, Chicago, Ill.) is placed in each reaction vessel. The synthesis is conducted on a Rainin Symphony Peptide Synthesizer. Analogs with a C-terminal amide are prepared using 75 mg (50 μmole) Rink Amide AM resin (Rapp Polymere, Tuebingen, Germany).

The following Fmoc amino acids are purchased from GlycoPep (Chicago, Ill.), and NovaBiochem (La Jolla, Calif.): Arg-2,2,4,6,7-pentamethyldihydrobenzofuran-5-sulfonyl (Pbf), Asn-trityl (Trt), Asp-β-t-Butyl ester (tBu), Asp-β-allyl ester (Allyl), Glu-δ-t-butyl ester (tBu), Glu-δ-allyl ester (Allyl), Gln-trityl (Trt), His-trityl (Trt), Lys-t-butyloxycarbonyl (Boc), Lys-allyloxycarbonyl (Aloc), Orn-allyloxycarbonyl (Aloc), Ser-t-butyl ether (OtBu), Thr-t-butyl ether (OtBu), Trp-t-butyloxycarbonyl (Boc), Tyr-t-butyl ether (OtBu).

Solvents dimethylformamide (DMF-Burdick and Jackson), N-methylpyrrolidone (NMP-Burdick and Jackson), dichloromethane (DCM-Mallinkrodt) are purchased from Mays Chemical Co. (Indianapolis, Ind.).

Hydroxybenzotrizole (HOBt), di-isopropylcarbodiimide (DIC), di-isopropylethylamine (DIEA), and piperidine (Pip) are purchased from Aldrich Chemical Co (Milwaukee, Wis.). Benzotriazole-1-yl-oxy-tris-(dimethylamino)-phosphoniumhexafluorophosphate (BOP) is obtained from NovaBiochem (San Diego, Calif.).

Cyclic VPAC2 receptor peptide agonists with a lactam bridge linking a lysine residue and an aspartic acid residue are prepared by selectively protecting the side chains of these residues with Aloc and Allyl, respectively. All other amino acids used in the synthesis are standard t-butyl side-chain protected Fmoc-amino acids.

All amino acids are dissolved in 0.3 M concentration in DMF. Three hours DIC/HOBt activated couplings are run after 20 minutes deprotection using 20% Piperidine/DMF. Each resin is washed with DMF after deprotections and couplings. After the last coupling and deprotection, the peptidyl resins are washed with DCM and are dried in vacuo in the reaction vessel. For the N-terminal acylation, four-fold excess of symmetric anhydride of the corresponding acid is added onto the peptide resin. The symmetric anhydride is prepared by diisopropylcarbodiimde (DIC) activation in DCM. The reaction is allowed to proceed for 4 hours and monitored by ninhydrin test. The Aloc and Allyl protecting groups are selectively removed and the cyclisation is carried out by activating the aspartic acid carboxyl group with BOP in the presence of DIEA. The peptide resin is then washed with DCM and dried in vacuo.

The cleavage reaction is mixed for 2 hours with a cleavage cocktail consisting of 0.2 mL thioanisole, 0.2 mL methanol, 0.4 mL triisopropylsilane, per 10 μL trifluoroacetic acid (TFA), all purchased from Aldrich Chemical Co., Milwaukee, Wis. If Cys is present in the sequence, 2% of ethanedithiol is added. The TFA filtrates are added to 40 mL ethyl ether. The precipitants are centrifuged 2 minutes at 2000 rpm. The supernatants are decanted. The pellets are resuspended in 40 mL ether, re-centrifuged, re-decanted, dried under nitrogen and then in vacuo.

0.3-0.6 mg of each product is dissolved in 1 mL 0.1% TFA/acetonitrile(ACN), with 20 μL being analyzed on HPLC [0.46×15 cm METASIL AQ C18, 1 mL/min, 45° C., 214 nM (0.2 A), A=0.1% TFA, B=0.1% TFA/50% ACN. Gradient=50% B to 90% B over 30 minutes].

Purifications are run on a 2.2×25 cm VYDAC C18 column in buffer A (0.1% trifluoroacteic acid in water, B: 0.1% TFA in acetonitrile). A gradient of 20% to 90% B is run on an HPLC (Waters) over 120 minutes at 10 mL/minute while monitoring the UV at 280 nm (4.0 A) and collecting 1 minute fractions. Appropriate fractions are combined, frozen and lyophilized. Dried products are analyzed by HPLC (0.46×15 cm METASIL AQ C18) and MALDI mass spectrometry.

### EXAMPLE 3

### In Vitro Potency

DiscoveRx: a CHO-S cell line stably expressing human VPAC2 receptor in a 96-well microtiter plate is seeded with 50,000 cells/well the day before the assay. The cells are allowed to attach for 24 hours in 200 μL culture medium. On the day of the experiment, the medium is removed. Also, the cells are washed twice. The cells are incubated in assay buffer plus IBMX for 15 minutes at room temperature. Afterwards, the stimuli are added and are dissolved in assay buffer. The stimuli are present for 30 minutes. Then, the assay buffer is gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate. In the case of VPAC1 and PAC1 receptors, CHO-PO cells are transiently transfected with human VPAC1 or PAC1 receptor DNA using commercially available transfection reagents (Lipofectamine from Invitrogen). The cells are seeded at a density of 10,000/well in a 96-well plate and are allowed to grow for 3 days in 200 mL culture medium. At day 3, the assay described above for the VPAC2 receptor cell line is performed.

Results for each agonist are the mean of two independent runs. VPAC1 and PAC1 results are only generated using the DiscoveRx assay. The typically tested concentrations of peptide are: 1000, 300, 100, 10, 1, 0.3, 0.1, 0.01, 0.001, 0.0001 and 0 nM.

Alpha screen: Cells are washed in the culture flask once with PBS. Then, the cells are rinsed with enzyme free dissociation buffer. The dissociated cells are removed. The cells are then spun down and washed in stimulation buffer. For each data point, 50,000 cells suspended in stimulation buffer are used. To this buffer, Alpha screen acceptor beads are added along with the stimuli. This mixture is incubated for 60 minutes. Lysis buffer and Alpha screen donor beads are added and are incubated for 60 to 120 minutes. The Alpha screen signal (indicative of intracellular cAMP levels) is read in a suitable instrument (e.g. AlphaQuest from Perkin-Elmer). Steps including Alpha screen donor and acceptor beads are performed in reduced light. The EC₅₀ for cAMP generation is calculated from the raw signal or is based on absolute cAMP levels as determined by a standard curve performed on each plate.

Results for each agonist are, at minimum, from two analyses performed in a single run. For some agonists, the results are the mean of more than one run. The tested peptide concentrations are: 10000, 1000, 100, 10, 3, 1, 0.1, 0.01, 0.003, 0.001, 0.0001 and 0.00001 nM.

The activity (EC₅₀ (nM)) for the human VPAC2, VPAC1, and PAC1 receptors is reported in Table 1.

TABLE 1

| | Human | Human | Human | Human |
|---|---|---|---|---|
| | VPAC2R: | VPAC2R: | VPAC1R: | PAC1R: |
| Agonist # | Alphascreen | DiscoveRx | DiscoveRx | DiscoveRx |
| VIP | 1.00 | 0.70 | 0.02 | >10 |
| PACAP-27 | 2.33 | 0.84 | 0.05 | 0.06 |
| P15 | n.d. | 2.33 | | |
| P16 | 53.10 | 6.21 | 1.1 | 38.6 |
| P17 | 3.19 | 1.20 | 52.7 | >300 |
| P57 | 0.09 | 0.14 | 24.3 | 563.0 |
| P77 | 1.29 | 0.94 | 46.3 | >1000 |
| P78 | 0.46 | 0.33 | 59.4 | >1000 |
| P86 | 694.7 | | | |
| P200 | 0.24 | | | |
| P225 | 0.09 | | | |
| P237 | 3.89 | | | |
| P248 | 0.82 | | | |
| P254 | 2.48 | | | |
| P256 | 1.94 | | | |
| P266 | 0.81 | | | |
| P267 | 0.18 | | | |
| P276 | 4.36 | | | |
| P280 | 0.15 | | | |
| P281 | 0.78 | | | |
| P287 | 0.15 | | | |
| P288 | 0.33 | | | |
| P303 | 0.09 | | | |
| P304 | 0.26 | | | |
| P310 | 0.13 | | | |
| P311 | 0.17 | | | |
| P312 | 0.11 | | | |
| P313 | 0.22 | | | |
| P360 | 0.17 | 0.04 | | |
| P361 | 0.16 | 0.04 | | |
| P374 | 0.21 | | | |
| P381 | 0.13 | | | |

EC₅₀ are in nM and either the result of a single determination or the average of two or more independent experiments. N.d. = not determined

### EXAMPLE 4

### Selectivity

Binding assays: Membrane prepared from a stable VPAC2 cell line (see Example 3) or from cells transiently transfected with human VPAC1 or PAC1 are used. A filter binding assay is performed using 125I-labeled VIP for VPAC1 and VPAC2 and 125I-labeled PACAP-27 for PAC1 as the tracers.

For this assay, the solutions and equipment include:

Presoak solution: 0.5% Polyethyleneamine in Aqua dest.

Buffer for flushing filter plates: 25 mM HEPES pH 7.4

Blocking buffer: 25 mM HEPES pH 7.4; 0.2% protease free BSA

Assay buffer: 25 mM HEPES pH 7.4; 0.5% protease free BSA

Dilution and assay plate: PS-Microplate, U form

Filtration Plate Multiscreen FB Opaque Plate; 1.0 μM Type B Glasfiber filter

In order to prepare the filter plates, the presoak solution is aspirated by vacuum filtration. The plates are flushed twice with 200 μL flush buffer. 200 μL blocking buffer is added to the filter plate. The filter plate is then incubated with 200 μL presoak solution for 1 hour at room temperature.

The assay plate is filled with 25 μL assay buffer, 25 μL membranes (2.5 μg) suspended in assay buffer, 25 μL compound (agonist) in assay buffer, and 25 μL tracer (about 40000 cpm) in assay buffer. The filled plate is incubated for 1 hour with shaking.

The transfer from assay plate to filter plate is conducted. The blocking buffer is aspirated by vacuum filtration and washed two times with flush buffer. 90 μL is transferred from the assay plate to the filter plate. The 90 μL transferred from assay plate is aspirated and washed three times with 200 μL flush buffer. The plastic support is removed. It is dried for 1 hour at 60° C. 30 μL Microscint is added. The count is performed.

The selectivity (IC₅₀) for human VPAC2, VPAC1, and PAC1 is reported in Table 2. Values reported are single results or the mean of two or more independent runs.

TABLE 2
Human receptor binding (IC50; nM)

| Agonist # | VPAC2 | VPAC1 | PAC1 |
|---|---|---|---|
| VIP | 5.06 | 3.3 | >1000 |
| PACAP-27 | 2.52 | 4 | 9.5 |
| P16 | 8.27 | 81.5 | n.d. |
| P86 | n.d. | >3000 | n.d. |
| P17 | 2.71 | 121.2 | >25000 |
| P57 | 0.24 | 108 | >25000 |
| P77 | 1.28 | 110.8 | n.d. |
| P78 | 0.44 | 103.7 | n.d. |
| P200 | 0.66 | 143.5 | n.d. |
| P225 | 0.2 | 143.3 | >25000 |
| P237 | 10.78 | 242.6 | >25000 |
| P248 | 7.64 | >3000 | >25000 |
| P254 | 20.51 | >3000 | 8587 |
| P256 | 11.09 | 5732.6 | 1026.5 |
| P266 | 21.71 | >3000 | >25000 |
| P267 | 1.12 | 350.4 | 2230.7 |
| P276 | 13.82 | 314.2 | 2486.5 |
| P280 | 0.59 | >3000 | >25000 |
| P281 | 12.17 | >3000 | >25000 |
| P287 | 0.16 | 200.5 | >25000 |
| P288 | 13.87 | 402.2 | >25000 |
| P303 | 0.13 | 214.6 | n.d. |
| P304 | 0.38 | >3000 | >25000 |
| P310 | 0.17 | >3000 | >25000 |
| P311 | 0.23 | >3000 | >25000 |
| P312 | 0.39 | 185.2 | >25000 |
| P313 | 0.69 | >3000 | >25000 |
| P359 | n.d. | >3000 | >25000 |
| P360 | 0.24 | >3000 | >25000 |
| P361 | 0.17 | >2000 | >25000 |
| P374 | 0.37 | >3000 | >25000 |
| P381 | 0.27 | n.d. | >25000 |

n.d. = not determined

Rat receptor selectivity was estimated by comparing functional potency (cAMP generation) in CHO-PO cells transiently expressing rat VPAC1 or rat VPAC2 receptors.

TABLE 3
In vitro potency using DiscoveRx (See Example 3). CHO-PO
cells are transiently transfected with rat VPAC1 or
VPAC2 receptor DNA. The activity (EC₅₀ in nM) for these
receptors is reported in the table below.

| Peptide # | rVPAC2 | rVPAC1 |
|---|---|---|
| VIP | 0.79 | 0.02 |
| PACAP-27 | n.d. | 0.07 |
| P15 | n.d. | 0.90 |
| P17 | 1.19 | 8.41 |
| P57 | 0.12 | 0.50 |
| P225 | n.d. | 0.75 |
| P237 | 3.70 | 8.01 |
| P248 | 0.25 | 25.87 |
| P254 | n.d. | 8.66 |
| P266 | 0.87 | 48.70 |
| P267 | n.d. | 0.12 |
| P280 | 0.09 | 0.62 |
| P303 | 0.05 | n.d. |
| P304 | 0.13 | 2.41 |
| P310 | 0.05 | 1.46 |
| P311 | 0.05 | 0.98 |
| P312 | 0.08 | 1.82 |
| P313 | 0.08 | 1.82 |
| P359 | 0.04 | n.d. |
| P360 | 0.04 | n.d. |
| P361 | 0.06 | 0.50 |
| P374 | 0.08 | 0.43 |
| P381 | 0.09 | 0.58 |

n.d. = not determined

### EXAMPLE 5

### In Vivo Assays

Intravenous glucose tolerance test (IVGTT): Normal Wistar rats are fasted overnight and are anesthetized prior to the experiment. A blood sampling catheter is inserted into the rats. The compound is given in the jugular vein. Blood samples are taken from the carotid artery. A blood sample is drawn immediately prior to the injection of glucose along with the compound. After the initial blood sample, glucose mixed with compound is injected intravenously (i.v.). A glucose challenge of 0.5 g/kg body weight is given, injecting a total of 1.5 mL vehicle with glucose and agonist per kg body weight. The peptide concentrations are varied to produce the desired dose in μg/kg. Blood samples are drawn at 2, 4, 6 and 10 minutes after giving glucose. The control group of animals receives the same vehicle along with glucose, but with no compound added. In some instances, a 30 minute post-glucose blood sample is drawn. Aprotinin is added to the blood sample (250 kIU/ml blood). The serum is then analyzed for glucose and insulin using standard methodologies.

Delayed IVGTT: IVGTT is performed as described above, making the following changes. After the initial blood sample, compound or vehicle is injected i.v. or s.c. glucose is injected i.v. 10-30 minutes later in a separate injection.

The assay uses a formulated and calibrated peptide stock in PBS. Normally, this stock is a prediluted 100 μM stock. However, a more concentrated stock with approximately 1 mg agonist per mL is used. The specific concentration is always known. Variability in the maximal response is mostly due to variability in the vehicle dose.

Protocol details are as follows:

| | |
|---|---|
| SPECIES/STRAIN/WEIGHT | Rat/Wistar Unilever/approximately |
| | 275-300 g |
| TREATMENT DURATION | Single dose |
| DOSE VOLUME/ROUTE | 1.5 mL/kg/iv |
| VEHICLE | 8% PEG300, 0.1% BSA in water |
| FOOD/WATER REGIMEN | Rats are fasted overnight prior to |
| | surgery. |
| LIVE-PHASE PARAMETERS | Animals are sacrificed at the end |
| | of the test. |
| IVGTT: Performed on rats | Glucose IV bolus: 500 mg/kg as 10% |
| (with two catheters, jugular | solution (5 mL/kg) at time = 0. |
| vein and carotid artery) of | Blood samplings (300 μL from |
| each group, under | carotid artery; EDTA as anticoagulant; |
| pentobarbital anesthesia. | aprotinin and PMSF as |
| | antiproteolytics; kept on ice): 0, 2, |
| | 4, 6, and 10 minutes. |
| | Parameter determined: Insulin. |
| TOXICOKINETICS | Plasma samples remaining after |
| | insulin measurements are kept |
| | at −20° C. and compound levels |
| | are determined. |

TABLE 4

| | Time | | | | |
|---|---|---|---|---|---|
| | between | % increase | % increase | % increase | % increase |
| | glucose & | AUC: Dose = | AUC: Dose = | AUC: Dose = | AUC: Dose = |
| Peptide | compound | 0.5 μg/kg | 3 μg/kg | 10 μg/kg | 30 μg/kg |
| P17 | 30 min | n.d. | n.d. | +187 | n.d. |
| P17* | 10 min | n.d. | +0.2 | +88 | +72 |
| P57* | 10 min | n.d. | n.d. | +39 | +129 |
| P248 | 0 min | +20 | n.d. | +125 | n.d |
| P280 | 0 min | +93** | n.d. | +233** | n.d |
| P281 | 0 min | n.d. | +155** | n.d. | +273** |
| P304 | 0 min | +212 | n.d. | +312 | n.d. |
| P304* | 10 min | n.d. | n.d. | +168 | n.d. |

*Compound given subcutaneously, n.d. = not determined, AUC = Area under curve (insulin, 0-10 or
**0-20 min after glucose)

Stepped-glucose infusion experiments. Similar to the IVGTT with the difference that glucose is infused in conscious rats instead of injected as a bolus. Two different glucose infusion rates are used, 5 and 15 mg/kg/min. Blood samples for insulin and glucose determinations are taken every 10 minutes and the infusion protocol consisted of 20 minute baseline infusion (no glucose) followed by a 30 min infusion period with the lower glucose rate and finally 30 minutes with the high glucose rate. The compound (e.g. P17) or vehicle is infused continuously by a subcutaneously implanted Alzet osmotic minipump (DURECT Corp., Cupertino, Calif., USA) for 72 h prior to the experiment. The assay uses a formulated and calibrated peptide stock in PBS. The pumps infuse solution by a constant rate (1 μl/h) and the dose is set by varying the peptide concentration of the PBS solution used. The specific concentration is always known.
In-vivo safety pharmacology. P17 is infused subcutaneously for 72 h using the Alzet minipump as described above for the stepped-glucose infusion experiment. Food consumption is recorded during day 1 to 3 of the infusion period and after 72 h the animals are sacrificed and the following blood parameters are analysed using conventional assays described in the art. In addition to the vehicle group two doses of P17 are given 3 and 9 μg/rat/h corresponding to approximately 8 and 26 μg/kg/h. Each group consists of 6 male Wistar rats. All values are given as mean and standard deviation (sd).

TABLE 5
Food consumption and body weight during P17 infusion (food
consumption was measured the last 48 h of the study)

| | Food | | Body weight | | Body weight | |
|---|---|---|---|---|---|---|
| | consumption | | day 0 | | day 3 | |
| | (grams/48 h) | | (grams) | | (grams) | |
| Group | mean | sd | mean | sd | mean | sd |
| Vehicle | 47.4 | 4.8 | 305.6 | 10.0 | 302.4 | 10.6 |
| P17 - 3 μg/rat/h | 39.4 | 5.0 | 306.1 | 6.0 | 312.3 | 8.1 |
| P17 - 9 μg/rat/h | 22.7 | 6.2 | 300.0 | 10.0 | 292.7 | 13.6 |

TABLE 6
Plasma lipids and total cholesterol after 72 h P17 infusion

| | Triglycerides | | Cholesterol | | Free fatty acids | |
|---|---|---|---|---|---|---|
| | (mg/dl) | | (mg/dl) | | (mg/dl) | |
| Group | mean | sd | mean | sd | mean | sd |
| Vehicle | 156.7 | 54.4 | 54.7 | 10.8 | 12.6 | 5.0 |
| P17 - 3 μg/rat/h | 64.8 | 32.0 | 46.7 | 4.5 | 5.8 | 2.7 |
| P17 - 9 μg/rat/h | 37.2 | 20.2 | 48.5 | 9.3 | 5.4 | 0.3 |

TABLE 7
Glucose, insulin & glucagon after 72 h P17 infusion

| | Glucose (mg/dl) | | Insulin (ng/ml) | | Glucagon (pg/ml) | |
|---|---|---|---|---|---|---|
| Group | mean | sd | mean | sd | mean | sd |
| Vehicle | 172.8 | 35.4 | 1.39 | 0.60 | 113.6 | 13.7 |
| P17 - 3 μg/rat/h | 180.8 | 41.8 | 0.84 | 0.27 | 112.3 | 47.3 |
| P17 - 9 μg/rat/h | 169.8 | 33.4 | 0.75 | 0.32 | 138.3 | 73.6 |

TABLE 8
Pituitary hormones & corticosterone after 72 h P17 infusion

| | | | Growth hormone | | Corticosterone | |
|---|---|---|---|---|---|---|
| | Prolactin (ng/ml) | | (ng/ml) | | (ng/ml) | |
| Group | mean | sd | mean | sd | mean | sd |
| Vehicle | 10.6 | 9.3 | 67.1 | 62.4 | 94.5 | 59.1 |
| P17 - 3 μg/rat/h | 12.8 | 12.9 | 60.6 | 116.4 | 125.8 | 123.5 |
| P17 - 9 μg/rat/h | 23.2 | 25.5 | 40.7 | 48.5 | 103.5 | 37.7 |

### EXAMPLE 6

### Serum Stability Studies

In order to determine the stability of VPAC2 receptor peptide agonists in rat serum, CHO-VPAC2 cells clone #6 (96 well plates/50,000 cells/well and 1 day culture), PBS 1× (Gibco), the peptides for the analysis in a 100 μM stock solution, rat serum from a sacrificed normal Wistar rat, aprotinin, and a DiscoveRx assay kit are obtained. The rat serum is stored at 4° C. until use and is used within two weeks.

On Day 0, two 100 μL aliquots of 10 μM peptide in rat serum are prepared by adding 10 μL peptide stock to 90 μL rat serum for each aliquot. 250 kIU aprotinin/mL is added to one of these aliquots. The aliquot is stored with aprotinin at 4° C. The aliquot is stored without aprotinin at 37° C. The aliquots are incubated for 18 hours.

On Day 1, after incubation of the aliquots prepared on day 0 for 24 hours, an incubation buffer containing PBS+1.3 mM CaCl₂, 1.2 mM MgCl₂, 2 mM glucose, and 0.25 mM IBMX is prepared. A plate with 11 serial 5× dilutions of peptide for the 4° C. and 37° C. aliquot is prepared for each peptide studied. 2000 nM is used as the maximal concentration if the peptide has an EC₅₀ above 1 nM and 1000 nM as maximal concentration if the peptide has an EC₅₀ below 1 nM from the primary screen (see Example 3). The plate(s) are washed with cells twice in incubation buffer. The plates are allowed to hold 50 μL incubation media per well for 15 minutes. 50 μL solution per well is transferred to the cells from the plate prepared with 11 serial 5× dilutions of peptide for the 4° C. and 37° C. aliquot for each peptide studied, using the maximal concentrations that are indicated by the primary screen, in duplicate. This step dilutes the peptide concentration by a factor of two. The cells are incubated at room temperature for 30 minutes. The supernatant is removed. 40 μL/well of the DiscoveRx antibody/extraction buffer is added. The cells are incubated on the shaker (300 rpm) for 1 hour. Normal procedure with the DiscoveRx kit is followed. cAMP standards are included in column 12. EC₅₀ values are determined from the cAMP assay data. The remaining amount of active peptide is estimated by the formula EC_{50, 4C}/EC_{50, 37C} for each condition.

TABLE 9

| | Rat Serum Stability (estimated |
|---|---|
| Peptide # | purity in % after 24 hours) |
| P15 | 17.2 |
| P17 | 38.8 |
| P57 | 26.8 |
| P77 | 27.6 |
| P78 | 24.2 |
| P225 | 36.6 |
| P237 | 48.4 |
| P248 | 70.1 |
| P254 | 42.8 |
| P256 | 38.8 |
| P266 | 93.2 |
| P267 | 16.5 |
| P276 | 7.8 |
| P280 | 39.8 |
| P281 | 73.5 |
| P287 | 17.7 |
| P288 | 0.5 |
| P303 | 32.3 |
| P304 | 84.7 |
| P310 | 20.9 |
| P311 | 89.2 |
| P312 | 36.3 |
| P313 | 79.1 |
| P359 | 85.7 |
| P360 | 76.3 |
| P361 | 74.4 |
| P374 | 98.6 |

Serum stability in human serum may also be determined using the above described protocol substituting rat serum for human serum (Eg. Sigma #H-4522, Lot #043 K0500). The estimated amounts of peptide (%) remaining after 24 h incubation at 37 C in human serum are listed in Table 11 below.

TABLE 11

| | Human Serum Stability (estimated |
|---|---|
| Peptide # | purity in % after 24 hours) |
| P17 | 63.8 |
| P361 | 88.0 |
| P374 | 99.1 |

Estimated 72 h serum stability values are obtained by using the protocol described for 24 h rat serum stability and by increasing the incubation time to 72 h. Estimates of 72 h rat serum stabilities are listed in Table 12.

TABLE 12

| | Rat Serum Stability (Estimated |
|---|---|
| Peptide | purity in % after 72 hours) |
| P15 | 13.5 |
| P17 | 18.1 |
| P57 | 8.7 |
| P225 | 1.6 |
| P237 | 4.2 |
| P248 | 51.4 |
| P254 | <0.1 |
| P256 | 88.1 |
| P266 | 44.8 |
| P281 | 66.1 |
| P288 | <0.1 |
| P304 | 37.7 |
| P311 | 22.3 |
| P313 | 39.0 |
| P359 | 73.8 |
| P360 | 47.1 |
| P361 | 44.1 |

### EXAMPLE 7

### Pharmacokinetic Assay

Healthy Fisher 344 rats (3 animals per group) are injected intravenously or subcutaneously with 100 μg compound/kg. Blood samples are drawn 5, 10, 20, 30, 45, 60, 120 and 240 min post dosing and the peptide content in plasma is analysed by ELISA detecting intact peptide by the aid of antibodies direct against the N- and C-termini of the peptide. PK parameters are then calculated using a model-independent method (WinNonlin Pro, Pharsight Corp., Mountain View, Calif., USA).

TABLE 13
PK parameters of compounds after i.v or s.c administration of
100 μg/kg compound. Mean and (SD) values for N = 3.

| | Exposure | Expo- | Exposure | Exposure | Exposure |
|---|---|---|---|---|---|
| Pep- | (Cmax; | sure | (clearance in | (Vdist in | (AUC₀₋ₗₐₛₜ, |
| tide | ng/ml) | (t½, min) | ml/(min*kg) | ml/kg) | ng*min/ml) |
| P57 | 151 | 12 | 36.5 | 365 | 2820 |
| i.v. | (30) | (6) | (6.2) | (65) | (480) |
| P57 | 14 | 12 | 194.1 | 3630 | 540 |
| s.c. | (2) | (<3) | (26.1) | (776) | (60) |
| P248 | 162.7 | 10.4 | 37.9 | 560 | 2625 |
| i.v. | (1.0) | (0.1) | (0.8) | (323) | (57) |
| P248 | 7.4 | 19.1 | 264.0 | 7062 | 340 |
| s.c. | (1.5) | (5.9) | (45.6) | (1406) | (41) |

Estimated sc bioavailabilities were: 19% (P57) & 13% (P248).

### EXAMPLE 8

### DPP-IV HPLC Assays

Part 1: Formulation of Selective VPAC2 Receptor Peptide Agonists:

Approximately 2 mg of lyophilized peptide is weighed and dissolved in approximately 1.6 mL de-ionized water. If the peptide does not dissolve, the pH is adjusted with 1M NaOH to between pH 10.0 and 10.5. After incubation at room temperature for 30 minutes, 1/10^{th} of the original volume 10×PBS is added. The pH is adjusted to between pH 7.2 and 7.6. The peptide solution is filtered through a 0.22 μm Millex-GV syringe filter (Millipore, Bedford Mass., USA). The peptide concentration is determined through absorption at 280 nm. The peptide concentration is then adjusted to 100 μM. The peptides are frozen at −20° C. for further use.

Part 2: In Vitro Incubation of Selective VPAC2 Receptor Peptide Agonists with Purified Dipeptidyl-Peptidase IV (DPP-IV):

The stability of selective VPAC2 receptor peptide agonists against proteolysis by DPP-IV is determined using 100 μL of a 100 μM peptide solution in 1×PBS. A 10 μL solution is removed and quenched with 40 μL of 0.1% trifluoroacetic acid (TFA)/20% acetonitrile (ACN). This solution (20 μL) is analyzed by reversed-phase HPLC. The reversed-phase analysis consists of a Zorbax 300SB-C8 column (3.5 micron, 4.6×50 mm, Alltech Associates, Inc., Deerfield Ill., USA) running a 15-40% B gradient over 15 minutes at 60° C. where A-buffer is 0.1% (v/v) TFA in water and B-buffer is 0.085% (v/v) TFA in ACN. The peak area is integrated. This peak area serves as an internal control as 100% intact peptide.

A 10 μL aliquot of a 1.12 mU/μL solution of DPP-IV (Sigma, St. Louis, Mo., USA) is added to 90 μL of a 100 μM solution of peptide, resulting in a substrate concentration of 90 μM peptide. The reaction mixture is then stored at 37° C. At various time-points, 10 μL of solution is removed, quenched with 40 μL 0.1% TFA/20% ACN, and analyzed by reversed-phase HPLC as described above. The remaining full length peptide concentration (nM) at each timepoint, except time=0, is calculated using

$$\frac{peak area [ time x ] * concentration [ t 0 ]}{peak area [ time 0 ] * 0.9}$$

For the time=0 timepoint, the concentration (nM) is calculated using the following formula:

$$\frac{peak area [ time x ] * initial substrate concentration [ 9 nM ]}{peak area [ time 0 ]}$$

TABLE 14
Concentration (nM) of Remaining Main Peak by RP-HPLC after
Incubation of Peptide with Purified Porcine DPP-IV

| Study | | | | | |
|---|---|---|---|---|---|
| Number | Sample | Time¹ = 0 | Time¹ = 2 | Time¹ = 6 | Time¹ = 24 |
| P17 | | 9.0 | 8.5 | 8.3 | 4.3 |

¹Time = Hours at 37° C.

Other modifications of the present invention will be apparent to those skilled in the art without departing from the scope of the invention.

1-39. (canceled)

40. A cyclic VPAC2 receptor peptide agonist, comprising the amino acid sequence shown in SEQ ID NO: 5:
His-Ser-Xaa₃-Ala-Val-Phe-Thr-Xaa8-Asn-Tyr(OMe)-
Thr-Xaa₁₂-Xaa₁₃-Xaa₁₄-Xaa₁₅-Xaa₁₆-Nle-Ala-Ala-
Xaa₂₀-Xaa₂₁-Tyr-Leu-Asn-Xaa₂₅-Xaa₂₆-Xaa₂₇-Xaa₂₈-
Xaa₂₉
wherein:
Xaa₃ is: Asp, or Glu;
Xaa₈ is: Asp, or Glu;
Xaa₁₂ is: Lys, Cys, hC, hR, Orn, or Dab;
Xaa₁₃ is: Leu, or Aib;
Xaa₁₄ is: Arg, or Aib;
Xaa₁₅ is: Lys, Orn, Dab, or Aib;
Xaa₁₆ is: Gln, Cys, or hC;
Xaa₂₀ is: Lys, hR, Orn, or Dab;
Xaa₂₁ is: Lys, Cys, hR, hC, Orn, or Dab;
Xaa₂₅ is: Ser. Cys, Asp, hC, or Glu;
Xaa₂₆ is: Leu, or Ile;
Xaa₂₇ is: Lys, hR, Orn, or Dab;
Xaa₂₈ is: Lys, Asn, hR, Gln, Aib, Orn, Dab, or Pro; and
Xaa₂₉ is: Lys, Orn, Dab, hR, or is absent; and
a C-terminal extension, wherein the N-terminus of said C-terminal extension is linked to the C-terminus of said peptide of SEQ ID NO: 5, wherein said C-terminal extension is selected from the group consisting of GGPSSGAPPPS (SEQ ID NO: 10), GGPSSGAPPPS—NH₂ (SEQ ID NO: 11), GGPSSGAPPPC(SEQ ID NO: 12), GGPSSGAPPPC—NH₂, (SEQ ID NO: 13), GRPSSGAPPPS (SEQ ID NO: 14), and GRPSSGAPPPS—NH₂ (SEQ ID NO: 15), and
wherein said cyclic VPAC2 receptor peptide of SEQ ID NO: 5 is cyclized by means of a lactam bridge formed by covalent attachment of the side chain of a Lys, Orn or Dab residue to the side chain of an Asp or Glu residue, or
wherein said cyclic VPAC2 receptor peptide of SEQ ID NO: 5 is cyclized by means of a disulfide bridge formed by covalent attachment of the side chain of a Cys or hC residue to the side chain of another Cys or hC residue, or
a pharmaceutically acceptable salt thereof.

41. The cyclic VPAC2 receptor peptide agonist according to claim 40, wherein said lactam bridge or said disulfide bridge is formed by the covalent attachment of the side chain of the residue at Xaaₙ to the side chain of the residue at Xaaₙ₊₄, wherein n is 12, 20, or 21

42. (canceled)

43. The cyclic VPAC2 receptor peptide agonist according to claim 40, further comprising an N-terminal modification, wherein said N-terminal modification is the addition of a group selected from the group consisting of: acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂.

44. The cyclic VPAC2 receptor peptide agonist according to claim 43, wherein said N-terminal modification is the addition of a group selected from of the group consisting of acetyl, hexanoyl, cyclohexanoyl, and propionyl.

45. The cyclic VPAC2 receptor peptide agonist according to claim 40, comprising the amino acid sequence shown in SEQ ID NO: 59:

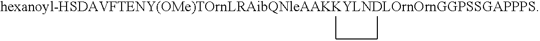

46. (canceled)

47. A method of treating non-insulin-dependent diabetes or insulin-dependent diabetes in a mammal in need thereof, comprising administering to said mammal an effective amount of said cyclic VPAC2 receptor peptide agonist according to claim 40.

48. The method of claim 47, wherein said mammal is a human.


SEQUENCE LISTING (59 sequences)

SEQ ID NO: 1 - protein, 40 aa, Artificial
  Synthetic Construct
XXXXXXTXXXTXXXXXXXXXXXXXXXXXXXXXXXXXXXXX

SEQ ID NO: 2 - protein, 13 aa, Artificial
  Synthetic Construct
XXXXXXXXXXXXX

SEQ ID NO: 3 - protein, 10 aa, Artificial
  Synthetic Construct
XXXXXXXXXX

SEQ ID NO: 4 - protein, 32 aa, Artificial
  Synthetic Construct
XXXXXXTXXXTXXXXXXXXXXXXXXXXXXXXX

SEQ ID NO: 5 - protein, 13 aa, Artificial
  Synthetic Construct
XXXXXXXXXXXXX

SEQ ID NO: 6 - protein, 10 aa, Artificial
  Synthetic Construct
XXXXXXXXXX

SEQ ID NO: 7 - protein, 29 aa, Artificial
  Synthetic Construct
HSXAVFTXNXTXXXXXXAAXXYLNXXXXX

SEQ ID NO: 8 - protein, 11 aa, Artificial
  Synthetic Construct
XXXXXXXXXXX

SEQ ID NO: 9 - protein, 9 aa, Artificial
  Synthetic Construct
XXXXXXXXX

SEQ ID NO: 10 - protein, 11 aa, Artificial
  Synthetic Construct
GGPSSGAPPPS

SEQ ID NO: 11 - protein, 11 aa, Artificial
  Synthetic Construct
GGPSSGAPPPS

SEQ ID NO: 12 - protein, 11 aa, Artificial
  Synthetic Construct
GGPSSGAPPPS

SEQ ID NO: 13 - protein, 11 aa, Artificial
  Synthetic Construct
GGPSSGAPPPS

SEQ ID NO: 14 - protein, 11 aa, Artificial
  Synthetic Construct
GGPSSGAPPPC

SEQ ID NO: 15 - protein, 11 aa, Artificial
  Synthetic Construct
GGPSSGAPPPC

SEQ ID NO: 16 - protein, 11 aa, Artificial
  Synthetic Construct
GRPSSGAPPPS

SEQ ID NO: 17 - protein, 11 aa, Artificial
  Synthetic Construct
GRPSSGAPPPS

SEQ ID NO: 18 - protein, 7 aa, Artificial
  Synthetic Construct
SRTSPPP

SEQ ID NO: 19 - protein, 7 aa, Artificial
  Synthetic Construct
SRTSPPP

SEQ ID NO: 20 - protein, 10 aa, Artificial
  Synthetic Construct
SSTSPRPPSS

SEQ ID NO: 21 - protein, 10 aa, Artificial
  Synthetic Construct
SSTSPRPPSS

SEQ ID NO: 22 - protein, 8 aa, Artificial
  Synthetic Construct
SRTSPPPX

SEQ ID NO: 23 - protein, 9 aa, Artificial
  Synthetic Construct
SRTSPPPKX

SEQ ID NO: 24 - protein, 39 aa, Artificial
  Synthetic Construct
HSDAVFTENYTKLRKQXAAKKYLNDLKKGGPSSGAPPPS

SEQ ID NO: 25 - protein, 39 aa, Artificial; identical to SEQ ID NO: 24
  Synthetic Construct

SEQ ID NO: 26 - protein, 38 aa, Artificial
  Synthetic Construct
HSDAVFTENYTKLRKQXAAKKYLNDLKKGGPSGAPPPS

SEQ ID NO: 27 - protein, 39 aa, Artificial; identical to SEQ ID NO: 24
  Synthetic Construct

SEQ ID NO: 28 - protein, 28 aa, Artificial
  Synthetic Construct
HSDAVFTENYTKLRKQXAAKXYLNXLKK

SEQ ID NO: 29 - protein, 39 aa, Artificial
  Synthetic Construct
HSDAVFTENYTKLRKQXAAKKYLNDLKKGGPSSGAPPPC

SEQ ID NO: 30 - protein, 39 aa, Artificial
  Synthetic Construct
HSDAVFTENYTKLRKQXAAKKYLNDLKQGGPSSGAPPPS

SEQ ID NO: 31 - protein, 39 aa, Artificial; identical to SEQ ID NO: 30
  Synthetic Construct

SEQ ID NO: 32 - protein, 39 aa, Artificial
  Synthetic Construct
HSDAVFTENYTKLRKQXAAKKYLNDLKQGGPSSGAPPPC

SEQ ID NO: 33 - protein, 39 aa, Artificial
  Synthetic Construct
HSDAVFTENYTKLXKQXAAKKYLNDLKKGGPSSGAPPPS

SEQ ID NO: 34 - protein, 39 aa, Artificial
  Synthetic Construct
HSDAVFTENYTKLXKQXAAKKYLNDLKQGGPSSGAPPPC

SEQ ID NO: 35 - protein, 39 aa, Artificial
  Synthetic Construct
HSDAVFTENYTKLXKQXAAKKYLNDLKKGGPSSGAPPPC

SEQ ID NO: 36 - protein, 39 aa, Artificial
  Synthetic Construct
HSDAVFTENYTKLXKQXAAKKYLNDLKQGGPSSGAPPPS

SEQ ID NO: 37 - protein, 40 aa, Artificial
  Synthetic Construct
HSDAVFTENYTKLRKQXAAKKYLNDLKQKGGPSSGAPPPC

SEQ ID NO: 38 - protein, 40 aa, Artificial
  Synthetic Construct
HSDAVFTENYTKLXKQXAAKKYLNDLKQKGGPSSGAPPPC

SEQ ID NO: 39 - protein, 39 aa, Artificial; identical to SEQ ID NO: 32
  Synthetic Construct

SEQ ID NO: 40 - protein, 39 aa, Artificial
  Synthetic Construct
HSDAVFTENYTKLRXQXAAKKYLNDLKKGGPSSGAPPPC

SEQ ID NO: 41 - protein, 39 aa, Artificial
  Synthetic Construct
HSDAVFTENYTKLRXQXAAKKYLNDLKKGGPSSGAPPPS

SEQ ID NO: 42 - protein, 40 aa, Artificial
  Synthetic Construct
HSDAVFTENYTKLXKQXAAKKYLNDLKQKGGPSSGAPPPS

SEQ ID NO: 43 - protein, 40 aa, Artificial
  Synthetic Construct
HSDAVFTENYTKLRKQXAAKKYLNDLKQKGGPSSGAPPPS

SEQ ID NO: 44 - protein, 39 aa, Artificial
  Synthetic Construct
HSDAVFTENYTKLRKQXAAKKYLNELKKGGPSSGAPPPS

SEQ ID NO: 45 - protein, 40 aa, Artificial
  Synthetic Construct
HSDAVFTENYTKLRKQXAAKKYLNDLKXKGGPSSGAPPPS

SEQ ID NO: 46 - protein, 40 aa, Artificial
  Synthetic Construct
HSDAVFTENYTKLRXQXAAKKYLNDLKXKGGPSSGAPPPS

SEQ ID NO: 47 - protein, 39 aa, Artificial
  Synthetic Construct
HSDAVFTENYTKLRKQXAAKKYLNDLKXGGPSSGAPPPS

SEQ ID NO: 48 - protein, 39 aa, Artificial
  Synthetic Construct
HSDAVFTENYTKLRXQXAAKKYLNDLKXGGPSSGAPPPS

SEQ ID NO: 49 - protein, 39 aa, Artificial; identical to SEQ ID NO: 47
  Synthetic Construct

SEQ ID NO: 50 - protein, 39 aa, Artificial; identical to SEQ ID NO: 48
  Synthetic Construct

SEQ ID NO: 51 - protein, 40 aa, Artificial
  Synthetic Construct
HSDAVFTENYTKLRXQXAAKKYLNDLKXKGGPSSGAPPPC

SEQ ID NO: 52 - protein, 40 aa, Artificial
  Synthetic Construct
HSDAVFTENYTKLRXQXAAKKYLNDLKXXGGPSSGAPPPS

SEQ ID NO: 53 - protein, 40 aa, Artificial
  Synthetic Construct
HSDAVFTENYTKLRXQXAAKKYLNDLXQXGGPSSGAPPPS

SEQ ID NO: 54 - protein, 39 aa, Artificial
  Synthetic Construct
HSDAVFTENYTKLRXQXAAKKYLNDLXXGGPSSGAPPPS

SEQ ID NO: 55 - protein, 39 aa, Artificial
  Synthetic Construct
HSDAVFTENYTXLRXQXAAKKYLNDLXXGGPSSGAPPPS

SEQ ID NO: 56 - protein, 39 aa, Artificial
  Synthetic Construct
HSDAVFTENYTXLRXQXAAXKYLNDLXXGGPSSGAPPPC

SEQ ID NO: 57 - protein, 39 aa, Artificial
  Synthetic Construct
HSDAVFTENYTXLRXQXAAXKYLNDLXXGGPSSGAPPPS

SEQ ID NO: 58 - protein, 39 aa, Artificial; identical to SEQ ID NO: 56
  Synthetic Construct

SEQ ID NO: 59 - protein, 39 aa, Artificial; identical to SEQ ID NO: 56
  Synthetic Construct